(19) United States
(12) Patent Application Publication
(10) Pub. No.: US 2024/0108674 A1
(43) Pub. Date: Apr. 4, 2024

(54) BERBERIS EXTRACT NANO-FORMULATION AND PROCESS OF PREPARATION THEREOF

(71) Applicant: PANJAB UNIVERSITY, CHANDIGARH, Chandigarh (IN)

(72) Inventors: Maninder KARAN, Chandigarh (IN); Karan VASISHT, Chandigarh (IN); Neetika SHARMA, Chandigarh (IN); Indu Pal KAUR, Chandigarh (IN); Vikas GAUTAM, Chandigarh (IN); Simarjot Kaur SANDHU, Chandigarh (IN); Jasmine KAUR, Chandigarh (IN)

(73) Assignee: PANJAB UNIVERSITY, CHANDIGARH, Chandigarh (IN)

(21) Appl. No.: 18/276,232

(22) PCT Filed: Feb. 8, 2022
(86) PCT No.: PCT/IN2022/050106

§ 371 Date: Aug. 7, 2023

(30) Foreign Application Priority Data
Feb. 8, 2021 (IN) 202111005303

(51) Int. Cl. A61K 36/29; A61K 9/51

(52) U.S. Cl. CPC A61K 36/29; A61K 9/5123; A61K 9/5146; A61K 9/5161; A61K 2236/15; A61K 2236/333; A61K 2236/51

(57) ABSTRACT

The present invention relates to a process for extraction and freeze-drying of a Berberis extract. More particularly, the invention provides an optimized process for freeze-drying/lyophilization to increase the solubility of the extract into the surfactants for enhanced incorporation into a nano-carrier system. Furthermore, the present invention also relates to a lipid nano-formulation of freeze-dried Berberis extract and process for preparing the same by high-pressure homogenization. The invention also relates to the lipid nano-formulation of freeze-dried Berberis extract for efficient drug delivery, for dermatological and cosmeceutical purposes, for treating diabetes and diabetes associated complications, as a wound healer, as an antimicrobial, as an anti-infective, as an anti-inflammatory agent and as an antioxidant.

### FIELD OF THE INVENTION

The present invention relates to a process for extraction and freeze-drying of Berberis extract. More particularly, the invention provides an optimized process for freeze-drying/lyophilization to increase the hydrophilicity or solubility of the extract into the surfactants for incorporation into a nano-carrier system. Furthermore, the present invention also relates to a lipid nano-formulation of freeze-dried Berberis extract and process for preparing the same by high-pressure homogenization. The invention also relates to the lipid nano-formulation of freeze-dried Berberis extract for efficient drug delivery, for dermatological and cosmeceutical purposes, for treatment of diabetes and diabetes associated complications, as a wound healer, as an antimicrobial, as an anti-infective, as an anti-inflammatory agent and as an antioxidant.

### BACKGROUND OF THE INVENTION

The Berberis extract is a multicomponent anti-infective and more particularly, an antibacterial substance credited with many more biologically useful activities. The conventional method of extraction and drying yields an extract with poor solubility in hydrophilic surfactants which limits the entrapment and its loading in making of nanostructured formulation. Therefore, the process of producing standardized Berberis extract was optimized and the extract was freeze-dried in the present invention.

U.S. Pat. No. 8,486,374 B2 relates to hygroscopic carriers and compositions, foamable carriers and foamable pharmaceutical and cosmetic compositions, wherein the solvent includes a polyethylene glycol or derivative or mixtures thereof or includes a propylene glycol derivative or combinations of polyethylene glycols with or without propylene glycol. In one or more embodiments, the vasoactive agent is a substance derived or extracted from herbs includes Achillea millefolium (yarrow), Allium sativum (garlic), Amoracia rusticana (horseradish), Berberis vulgaris (barberry), Cimicifuga racemose (black cohosh), Coleus forskohlii (coleus), Coptis (golden thread) etc.

The drawback of this prior art is that it involves preparation of waterless composition with 70-99% of polyethylene glycol and it does not contain any hydrophobic component.

Therefore, there is a need of a process that involves preparation of aqueous dispersion of lipid nano-formulation for making it easily washable and within the permissible limits of polyethylene glycol. Also, there is a requirement of the controlled release of the extract.

KR100998534B1 describes barberry trapped in the water-soluble gelatin. Berberis koreana is used to form nanoparticles and its manufacturing method involves adjusting the dissolution rate within the wood cell extract for reducing cytotoxicity.

Therefore, there is a requirement of a process that involves preparation of aqueous dispersion of lipid nano-formulation which is easy to use, has high drug loading of the extract into nanocarrier system for providing controlled release with increased bioavailability and thereby increased efficacy and safety.

Xue et al. 2015 (Berberine-loaded solid lipid nanoparticles are concentrated in the liver and ameliorate hepatosteatosis in db/db mice. Int J Nanomedicine. 2015 Aug. 5; 10: 5049-57. doi: 10.2147/IJN.S84565.) provides that berberine (BBR), an isolate of Berberis extract, shows very low plasma levels after oral administration due to its poor absorption by the gastrointestinal tract. It has also been demonstrated that BBR showed increased gastrointestinal absorption and enhanced antidiabetic effects in db/db mice model after being entrapped into solid lipid nanoparticles.

The drawback of this prior art is that it involves formulation of a pure compound berberine with low drug loading of only 4.2%.

Therefore, there is a need of a process for preparing the nano-formulation to increase high drug loading.

Rad et al., 2017 (Rad et al. Toxicology effects of Berberis vulgaris (barberry) and its active constituent, berberine: a review. Iranian Journal of Basic Medical Sciences, 2017; 20(5): 516-529. doi: 10.22038/ijbms.2017.8676) provides classified information about the toxicity of B. vulgaris and berberine in different conditions consist of acute, subacute, sub-chronic and chronic state. Besides, it discusses the cytotoxicity, genotoxicity, mutagenicity, and carcinogenicity of B. vulgaris and berberine as well as developmental toxicity and clinical studies.

Hee et al., 2015 (Development and factorial design of glyceryl tristearate based solid lipid nanoparticles containing berberine: Rapports De Pharmacie. 2016 Vol 2(1): 199-210.) talks about a nanoparticle formulation prepared by hot solvent evaporation method. A 3-factor, 2-level Box-Behnken design was used to optimize the process parameters including Triglyceride (A), Tween 20 (B) and Span 40 (C).

The prior art involves formulation of Berberine HCl into nanodispersion. Nanodispersion was prepared by hot solvent evaporation method. Method of preparation is for a pure molecule and involves the use of organic solvents.

Therefore, there is a requirement of a composition that involves standardized plant extract. Also, there is a requirement of a process for preparing the nano-formulation which is industrially viable, environment friendly and without using any organic solvents.

Wang et al., 2014 (Enhancing the antitumor activity of berberine hydrochloride by solid lipid nanoparticle encapsulation, AAPS PharmSciTech. 2014 August; 15(4): 834-844. doi: 10.1208/s12249-014-0112-0) refers to a solid lipid nanoparticle-based system which was developed for efficient incorporation and persistent release of berberine hydrochloride. Berberine HCl is added to solid lipid before melting and homogenisation. It involves addition of Tween 80 to lipidic phase. Drug loading is reported to be 2.84%.

This prior art involves formulation of pure compound with very low drug loading of 2.84%.

Therefore, there is need of a process for preparing the lipid nano-formulation using standardized Berberis plant extract with increased drug loading. Also, there is a need of a process which complement the effects of berberine, improves bioavailability, with reduction in dose to make it safer and profitable.

The prior art suffers from drawbacks such as low drug loading, poor absorption and use of organic solvents. Therefore, there is a need of a simple and convenient process for preparing lipid nano-formulation loaded with Berberis extract to circumvent its poor solubility and bioavailability by presenting it in a solubilized form as aqueous dispersion.

### Objective of the Invention

The main objective of present invention is to increase the drug loading and entrapment of Berberis extract into a nano-formulation by increasing its solubility.

A further objective of the present invention is to provide a process with optimized conditions for extraction and lyophilization/freeze-drying of the Berberis extract to increase its solubility.

Another objective of the present invention is to increase the bioavailability and protect the Berberis extract against hydrolytic and photodegradation by providing a lipid-based nano-formulation of freeze-dried Berberis extract.

A preferred objective of the present invention is to provide a lipid nano-formulation of freeze-dried Berberis extract which shows enhanced oral bioavailability or enhanced penetration through biological membrane and achieves effective delivery of the freeze-dried Berberis extract to epidermal, dermal, and subcutaneous layers following topical application.

An objective of the present invention is to provide a method for preparation of freeze-dried Berberis extract loaded lipid nano-formulation.

Yet another objective of the present invention is to incorporate the freeze-dried Berberis extract loaded lipid nano-formulation into semisolid, gel base, liquid solutions, liquid or aerosolized spray or film or solid dosage form or nasal spray or powder form to increase applicability while ensuring stability.

Yet another objective of the present invention is to provide solid lipid nanoparticles prepared by the process in the form of a dispersion which can therefore be used for efficient oral, parenteral, ocular, dental, buccal, intranasal, vaginal, rectal, otic, transdermal and topical delivery.

Yet another objective of the present invention is to provide a lipid nano-formulation which is used for dermatological and cosmeceutical purposes, for treating diabetes and more specifically diabetes associated complications, as a wound healer, as an antimicrobial, as an anti-infective, as an anti-inflammatory agent and as an antioxidant.

### SUMMARY OF INVENTION

The present invention provides a lipid nano-formulation and a process for preparing the same which address the aforesaid drawbacks of the prior arts. The process of the present invention involves optimization of the extraction process and enrichment of the Berberis extract for obtaining uniform quality of the extract. This extract with poor solubility profile was subjected to various processes for improvement in structural stability and solubility for the purpose of formulation development. Of various techniques, the conditions of freeze-drying/lyophilisation were optimized to achieve targeted results. The freeze-dried extract showed more than 10-fold increase in solubility in hydrophilic surfactants than the extract prepared by conventional methods. The resultant freeze-dried Berberis extract used in nanoformulation was prepared using (i) optimized extraction procedure and (ii) optimized combination of conditions of drying by freeze-drying/lyophilization to increase its hydrophilicity or solubility into the surfactants for incorporation into a nanocarrier system.

The extract loading capacity was increased significantly by more than 7-fold using nanocarrier system. The developed solid lipid nanoparticles had a drug loading of 25% with respect to the lipid phase with more than 90% entrapment and small particle size. The efficient entrapment of the extract within the core of these nanoparticles in a solubilized form increases its efficacy. The lipid core will provide protection to the extract against oxidation, and hydrolytic and photo-degradation in addition to providing freeze-dried Berberis extract in a bioavailable and controlled release manner. Biocompatible, cheap, easily available components including a lipid, non-ionic surfactants and surfactant supporting agents/cosolvents are used. The formulation is an aqueous dispersion of freeze-dried Berberis extract which is water soluble and washable. The freeze-dried Berberis extract SLNs are prepared using high pressure homogenization method which is industrially viable and without using any organic solvents. The developed formulation was tested for its wound healing potential in acute and chronic diabetic wounds. It showed significant faster healing of wounds in acute as well as chronic wounds.

Thus, increase in the extract solubility into hydrophilic solvents resulted in increased drug loading which in turn increased drug delivery and maximum therapeutic efficacy was achievable for faster healing of wounds consuming lower quantities of the drug material.

Accordingly, the present invention provides a lipid nano-formulation comprising freeze-dried Berberis extract and at least one lipid.

In one of the aspects of the present invention, the formulation comprises:
a) freeze-dried Berberis extract in a range of 0.5-10 percent by weight,
b) at least one lipid in a range of 2-12 percent by weight,
c) at least one surfactant in a range of 2-10 percent by weight,
d) at least one cosurfactant in a range of 0.2-1 percent by weight,
e) at least one solubilizing agent in a range of 4-10 percent by weight,
f) water,
wherein the percent by weight is with reference to the weight of the total formulation.

In another aspect, the freeze-dried Berberis extract is an alcoholic, hydroalcoholic or aqueous extract of freeze-dried Berberis.

In yet another aspect, the lipid component is selected from the group consisting of Compritol 888 ATO, stearic acid, glycerol monostearate, and Precirol.

In a further aspect, the solubilizing agent is selected from polyethylene glycol (PEG), polyvinylpyrrolidone (PVP), polyvinyl alcohol (PVA), glycerol, transcutol, labrafac, gelucire, hydrogenated vegetable glycerides, glyceryl citrate, glyceryl lactate, glyceryl lincolate, glyceryl oleate, polyglyceryl-4-cocoate, polyglyceryl-3-caprate and caprylate and their derivatives, polypropylene glycol, and propylene glycol. In a preferred embodiment of the present invention, the solubilizing agent is PEG 400.

In one aspect, the surfactant is selected from the group consisting of ethylene oxide copolymers, propylene oxide copolymers, poloxamers, sorbitan ethylene oxide/propylene oxide copolymers, polysorbate 20, polysorbate 60, polysorbate 80, sorbitan esters, span 20, span 40, span 60, span 80, alkyl aryl polyether alcohol polymers, tyloxapol, bile salts, cholate, glycocholate, taurocholate, taurodeoxycholate, gemini surfactants, alcohols, diethylene glycol monoethyl ether, propanediol, capryl glucoside, decyl glucoside, kolliwax or mixtures thereof. In a preferred embodiment of the present invention, the surfactant is polysorbate 80 also known as Tween 80.

In an aspect, cosurfactant is selected from the group consisting of phospholipon 90 G, soy lecithin, egg lecithin, phosphatidylcholine, cholate, glycocholate, taurocholate, taurodeoxycholate, or mixtures thereof. In a preferred embodiment of the present invention, the cosurfactant is soy lecithin or phospholipon 90.

In a preferred aspect, the formulation comprises:
a) freeze-dried Berberis extract in a range of 0.5-10 percent by weight,
b) Compritol in a range of 2-12 percent by weight,
c) Polysorbate 80 in a range of 2-10 percent by weight,
d) Phospholipon 90 G in a range of 0.2-1 percent by weight,
e) PEG 400 in a range of 4-10 percent by weight,
f) water,
wherein the percent by weight is with reference to the weight of the total formulation.

In a detailed aspect, the nano-formulation has a freeze-dried Berberis extract loading of 20 to 30% with respect to the lipid phase, freeze-dried Berberis extract entrapment efficiency in a range of 88-92%, particle size in a range of 50-500 nm and PDI in a range of 0.27-0.29.

In another preferred aspect, the lipid nano-formulation is solid lipid nanoparticles.

In one aspect, the lipid nano-formulation is combined with a suitable excipient to result in a gel, a hydrogel, an organogel, a syrup, a paste, a cream, a liquid wash, a facewash, a mouthwash, an oral rinse, an ointment, a liquid ampoule, a dispersion, a nasal drops or spray, an aerosol spray, a powder, an orthotic aid, a liquid oral formulation, a facemask, a film, an implant, a tablet, lozenges, capsules, suppositories, pessaries, patches and gummies. Further, the SLNs are in the form of a dispersion for oral, parenteral, ocular, intranasal, dental, buccal, vaginal, rectal, otic, transdermal, and topical delivery.

In yet another aspect, the formulation is as and when used for treating diabetes and diabetes associated complications.

In yet another aspect, the formulation is used as a wound healer, as an antimicrobial, as an anti-infective, as an anti-inflammatory agent and as an antioxidant.

In yet another embodiment, SLNs exhibit any therapeutic property as shown by Berberis extract.

The present invention also provides a process for preparation of the lipid nano-formulation of freeze-dried Berberis extract comprising:
a) preparing a lipid phase by melting one or more lipid at a temperature at least equal to the melting point of said one or more lipid,
b) separately preparing an aqueous phase by heating at least one emulsifier, at least one surfactant, and water at a temperature at least equal to said melting point of said one or more lipid of step a),
c) dissolving freeze-dried Berberis extract with at least one solubilizing agent to obtain a solution of freeze-dried extract of Berberis,
d) adding the solution of freeze-dried extract of Berberis to the lipid phase to obtain a hot lipid phase,
e) blending the hot lipid phase and the aqueous phase at a high speed of 5000-20,000 rpm for 5-30 minutes to obtain a hot emulsion,
f) subjecting hot emulsion obtained in step e) to two to six cycles of homogenization at 500-1200 bars pressure to obtain the lipid nano-formulation which is finally refrigerated after cooling to room temperature.

In yet a further aspect, there is provided a process for preparing the freeze-dried Berberis extract comprising:
i. cutting and crushing plant parts to obtain a moderately fine powder of Berberis,
ii. packing the powder obtained in step i. in a polyethylene bag and storing in a freezer at −20° C. to obtain frozen Berberis powder,
iii. carrying out reflux extraction of the frozen Berberis powder with an extraction solvent at 50-90° C. for 30 minutes to 8 hours,
iv. filtering the extracts through Whatman filter paper and concentrating the extract to dryness,
v. standardizing the extract by analysing the marker content of berberine, palmatine, jatrorrhizine and magnoflorine, producing fingerprint profiles and ATR fingerprints to obtain a standardized extract of Berberis,
vi. pre-treating the standardized extract by concentrating the extract at a temperature in a range of 19° C. to 70° C. and pressure in a range of 10 mmHg/torr to 30 mmHg/torr for reducing extract volume to about 1 to 25 mL to obtain concentrated extract,
vii. freezing the extract obtained in step vi. at a temperature in a range of −30 to −50° C. for 8 to 24 hours with 1-10 mm thickness of concentrated extract,
viii. primary drying the extract obtained in step vii. at a temperature in a range of −45 to −50° C. and pressure in a range of 25 to 30 mTorr for 15 to 20 hours, and
ix. secondary drying the extract obtained in step viii. at a temperature in a range of −45 to −60° C. and pressure in a range of 25 to 30 mTorr for 15 to 48 hours to obtain the freeze-dried Berberis extract.

In a preferred aspect, the plant part used in step i. is a stem, root, or a mixture thereof.

In another preferred aspect, the plant part is from one of the species of genus Berberis or Mahonia.

In a further aspect, the extraction solvent of step iii. is an organic or inorganic solvent.

In one aspect, the extraction solvent of step iii. is an alcohol, water or a mixture of alcohol and water.

### BRIEF DESCRIPTION OF THE DRAWINGS

FIG. 1: illustrates optical microscopy images of freeze-dried and vacuum oven-dried Berberis extract;

FIG. 2: illustrates the IR spectra of A. vacuum oven-dried extract and B. freeze-dried Berberis extract;

FIG. 3: illustrates the DSC curve of vacuum oven-dried Berberis extract and freeze-dried Berberis extract;

FIG. 4: illustrates the overlap diffraction spectra of vacuum-oven dried Berberis extract (RD) over freeze-dried Berberis extract (FD);

FIG. 5: illustrates the TGA of A. vacuum oven-dried Berberis extract and B. freeze-dried Berberis extract;

FIG. 6: illustrates the HSM images of vacuum oven-dried Berberis extract at different temperatures;

FIG. 7: illustrates the HSM images of freeze dried Berberis extract at different temperatures;

FIG. 8: illustrates optical microscopy images of freeze-dried Berberis extract loaded Solid lipid nanoparticles (SLNs);

FIG. 9: illustrates Field emission scanning electron microscopy (FESEM) images of freeze-dried Berberis extract loaded SLNs;

FIG. 10: illustrates the particle size analysis of freeze-dried Berberis extract loaded SLNs;

FIG. 11: illustrates the In vitro release profile of freeze-dried Berberis extract SLNs;

FIG. 12: illustrates the FT-IR spectra of a) freeze-dried Berberis extract, b) freeze-dried Berberis extract loaded SLNs, c) Compritol® 888 ATO, d) phospholipon 90 G, e) Physical mixture of Compritol® 888 ATO and PEG 400 (melted and solidified);

FIG. 13: illustrates the DSC thermograms of a) freeze-dried Berberis extract, b) freeze-dried Berberis extract loaded SLNs, c) Compritol® 888 ATO;

FIG. 14: illustrates the PXRD of a) Compritol® 888 ATO b) Compritol® 888 ATO melted with PEG 400 c) phospholipon 90 G d) freeze-dried Berberis extract loaded SLNs;

FIG. 15: illustrates the rheological profile of freeze-dried Berberis extract loaded SLNs gel depicting the relationship between shear rate, viscosity and shear stress;

FIG. 16: illustrates the texture analysis plot of freeze-dried Berberis extract loaded SLNs gel;

FIG. 17: illustrates the In vitro release profile of freeze-dried Berberis extract loaded SLNs gel (BSA SLNs) and freeze-dried Berberis extract gel (BSA);

FIG. 18: demonstrates the rabbit skin before and after application (72 hours) of freeze-dried Berberis extract loaded SLNs;

FIG. 19: illustrates the effect of different treatment groups on the excision diabetic wound on different days;

FIG. 20: illustrates the effect of different treatment groups on the incision diabetic wound on different days;

FIG. 21: illustrates the effect of different treatment and control groups on protein content in a diabetic wound excision model;

FIG. 22: illustrates the effect of different treatment and control groups on protein content in a diabetic wound incision model;

FIG. 23: illustrates the effect of different treatment and control groups on AChE activity in a diabetic wound excision model;

FIG. 24: illustrates the effect of different treatment and control groups on AChE activity in a diabetic wound incision model;

FIG. 25: illustrates the effect of different treatment and control groups on SOD levels in a diabetic wound excision model;

FIG. 26: illustrates the effect of different treatment and control groups on GSH levels in a diabetic wound excision model;

FIG. 27: illustrates the effect of different treatment and control groups on catalase levels in a diabetic wound excision model;

FIG. 28: illustrates the effect of different treatment and control groups on SOD levels in a diabetic wound incision model;

FIG. 29: illustrates the effect of different treatment and control groups on GSH levels in a diabetic wound incision model;

FIG. 30: illustrates the effect of different treatment and control groups on catalase levels in a diabetic wound incision model;

FIG. 31: illustrates the effect of different treatment and control groups on LPO levels in a diabetic wound excision model;

FIG. 32: illustrates the effect of different treatment and control groups on LPO levels in a diabetic wound incision model;

FIG. 33: provides photomicrographs of histopathological study of skin sections after staining with haematoxylin eosin in different treatment and control groups;

FIG. 34: provides photomicrographs of histopathological study of skin sections after staining with haematoxylin eosin in different treatment and control groups;

FIG. 35: illustrates the anti-inflammatory activity of A. prednisolone and Berberis extract B. diclofenac sodium and Berberis extract.

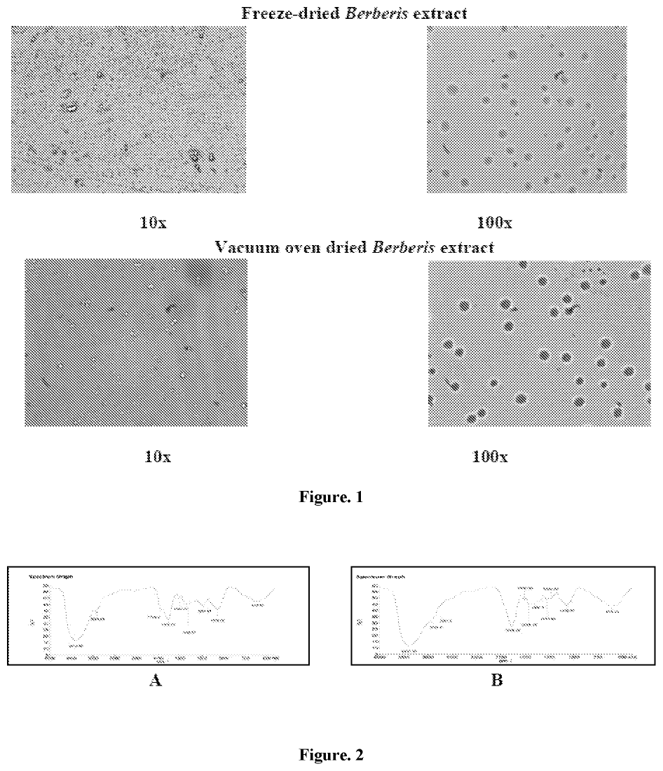

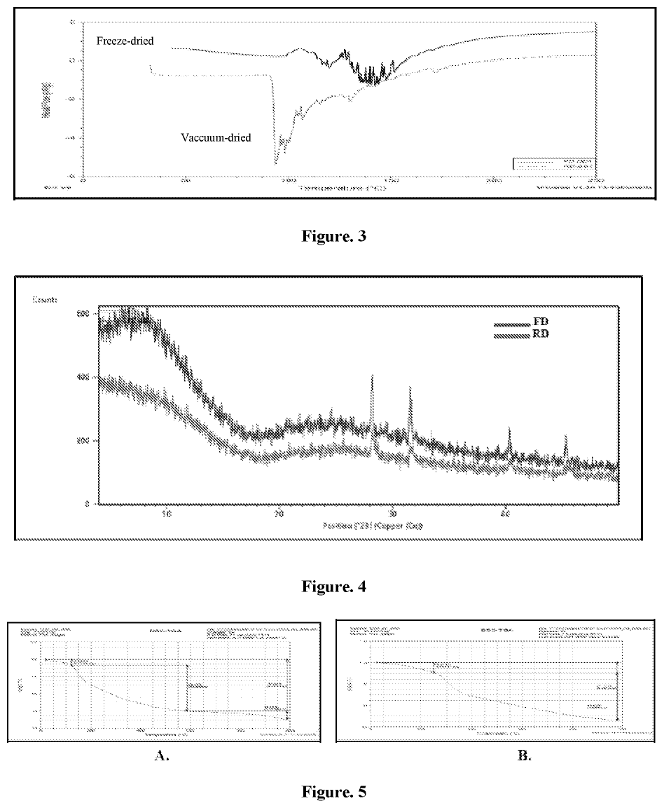

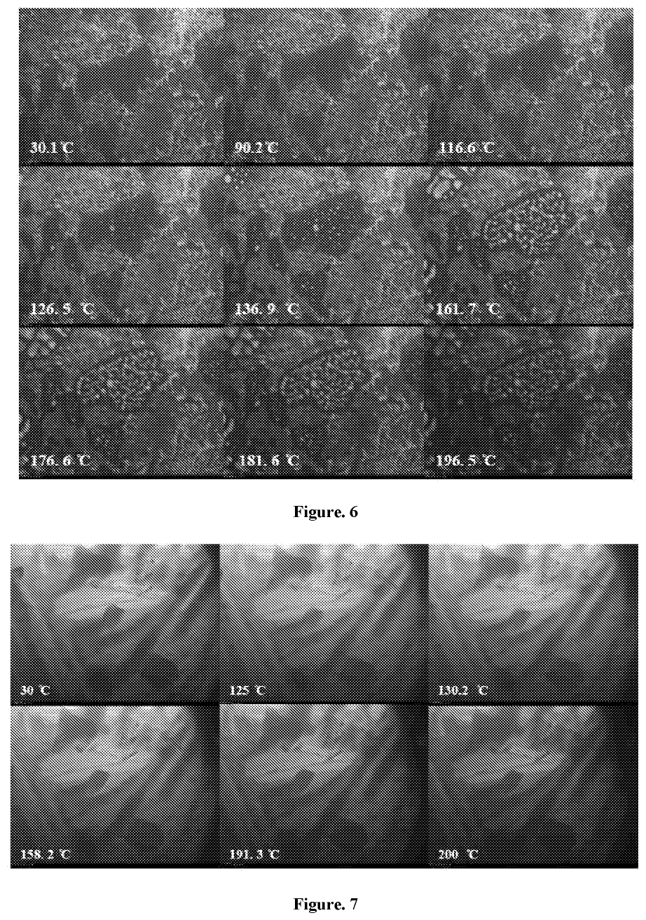

### DESCRIPTION OF THE INVENTION

Those skilled in the art will be aware that the disclosure is subject to variations and modifications other than those specifically described. It is to be understood that the disclosure includes all such variations and modifications. The disclosure also includes all such steps of the process, features of the product, referred to or indicated in this specification, individually or collectively, and any and all combinations of any or more of such steps or features.

The present invention outlines a unique method of preparation of extract where 10-fold increase in solubility of the extract in hydrophilic surfactants and more than 7-folds increase in extract loading capacity in the nano-formulation was achieved over the reported methods. The developed nano-formulation of the extract was more effective and guarded the extract against possible degradation by oxidation, hydrolytic and photodegradation. The loading capacity of the resultant extract was increased significantly by more than 7-folds in the nano-formulation because of increased solubility of the extract in hydrophilic solvents. The efficient entrapment of the extract within the core of these nano-formulation in a solubilized form increased the extract loading and efficacy. The lipid core provides the protection to the extract against degradation besides releasing it in a controlled manner and more bioavailable form. Biocompatible, cheap, easily available excipients including a lipid, non-ionic surfactants and surfactant supporting agents were used. The formulation is an aqueous dispersion of freeze-dried Berberis extract which is water soluble and washable. The developed formulation showed significantly faster healing of wounds in acute and chronic wounds especially in wounds associated with diabetes complications. Thus, increase in the extract solubility in hydrophilic solvents resulted in increased drug loading which in turn increased drug delivery and therapeutic efficacy for faster healing of wounds consuming lower quantities of the drug material.

The extraction and the drying method were optimized to yield the desired traits of the extract. The assay method of four makers in the extract was developed to ensure consistent and uniform quality of the extract. The conventionally prepared and oven-dried extract showed poor hydrophilicity and the method of drying was suitably modified to improve solubility of the extract for the purpose of formulation development. Of the various techniques of drying, freeze-drying/lyophilisation under specific conditions provided the targeted results. This included: 1) Pretreatment prior to lyophilisation for removing extraction solvent by one of the several available conventional techniques. 2) Identifying and optimizing freezing parameters of temperature, time, and thickness of sample in a circular tray. Optimized freezing conditions were: −40° C., 14 h, 5 mm thickness of sample in a dish. 3) Drying (ice sublimation) under a set of conditions of temperature, vapour pressure and time at: −50° C., 29.5 mTorr/0.039330 Bar, 18 h. The thermodynamic studies of the yielded extract revealed that under these conditions a change in physical nature of the extract from crystalline to amorphous is affected which also controlled the stickiness of the extract.

Accordingly, the present disclosure provides a lipid nano-formulation comprising freeze-dried Berberis extract and at least one lipid.

In a preferred embodiment, there is provided a lipid nano-formulation, comprising:
a) freeze-dried Berberis extract in a range of 0.5-10 percent by weight,
b) at least one lipid in a range of 2-12 percent by weight,
c) at least one surfactant in a range of 2-10 percent by weight,
d) at least one cosurfactant in a range of 0.2-1 percent by weight,
e) at least one solubilizing agent in a range of 4-10 percent by weight,
f) water,
wherein the percent by weight is with reference to the weight of the total formulation.

In a preferred embodiment, the freeze-dried Berberis extract is an alcoholic, hydroalcoholic or aqueous extract of freeze-dried Berberis.

In another preferred embodiment, the lipid component is selected from the group consisting of Compritol 888 ATO, stearic acid, glycerol monostearate, and Precirol. Compritol 888 ATO is a blend of different esters of behenic acid with glycerol mixture of glycerol monobehenate (12-18% w/w), glycerol dibehenate (45-54% w/w) and glycerol tribehenate (28-32% w/w). Phospholipon 90 G is pure phosphatidylcholine stabilized with 0.1% ascorbyl palmitate. Precirol ATO 5 is glyceryl palmitostearate containing palmitic acid, stearic acid and glycerol; is a mixture of mono-, di-, and triglycerides of C₁₆ and C₁₈ fatty acids.

In an embodiment, the solubilizing agent is selected from PEG 400, polyethylene glycol, PVP, PVA, glycerol, transcutol, labrafac, gelucire, hydrogenated vegetable glycerides, glyceryl citrate, glyceryl lactate, glyceryl lincolate, glyceryl oleate, polyglyceryl-4-cocoate, polyglyceryl-3-caprate and caprylate and their derivatives, polypropylene glycol, and propylene glycol. In a preferred embodiment of the present invention, the solubilizing agent is PEG 400.

In an embodiment, the surfactant is selected from the group consisting of ethylene oxide copolymers, propylene oxide copolymers, poloxamers, sorbitan ethylene oxide/propylene oxide copolymers, polysorbate 20, polysorbate 60, polysorbate 80, sorbitan esters, span 20, span 40, span 60, span 80, alkyl aryl polyether alcohol polymers, tyloxapol, bile salts, cholate, glycocholate, taurocholate, taurodeoxycholate, gemini surfactants, alcohols, diethylene glycol monoethyl ether, propanediol, capryl glucoside, decyl glucoside, kolliwax or mixtures thereof. In a preferred embodiment of the present invention, the surfactant is polysorbate 80.

In yet another embodiment, co-surfactant is selected from the group consisting of soy lecithin, egg lecithin, phosphatidylcholine, cholate, glycocholate, taurocholate, taurodeoxycholate, or mixtures thereof. In a preferred embodiment of the present invention, the cosurfactant is soy lecithin or phospholipon 90.

In a preferred embodiment, the lipid nano-formulation comprises:
a) freeze-dried Berberis extract in a range of 0.5-10 percent by weight,
b) Compritol in a range of 2-12 percent by weight,
c) Polysorbate 80 in a range of 2-10 percent by weight,
d) Phospholipon 90 G in a range of 0.2-1 percent by weight,
e) PEG 400 in a range of 4-10 percent by weight,
f) water,
wherein the percent by weight is with reference to the weight of the total formulation.

In an embodiment, the lipid nano-formulation has high Berberis extract loading of 20-30% with respect to the lipid phase; Berberis extract entrapment efficiency in a range of 88-92%, particle size in a range of 50-500 nm and PDI in a range of 0.27-0.29.

In a preferred embodiment, the lipid nano-formulation has high Berberis extract loading of 25% with respect to the lipid phase, Berberis extract entrapment efficiency of 90.56%±1.25, particle size in a range of 178.4 nm and PDI of 0.289.

In one embodiment, the lipid nano-formulation is selected from the group consisting of liposomes, solid lipid nanoparticles (SLNs) or micelles. In one embodiment, the lipid nano-formulation is solid lipid nanoparticles.

In a preferred embodiment, the disclosure provides a process for preparation of lipid nano-formulation of freeze-dried Berberis extract comprising:
a) preparing a lipid phase by melting one or more lipid at a temperature at least equal to the melting point of said one or more lipid,
b) separately preparing an aqueous phase by heating at least one emulsifier, at least one surfactant, and water at a temperature at least equal to said melting point of said one or more lipid of step a),
c) dissolving freeze-dried Berberis extract with at least one solubilizing agent to obtain a solution of freeze-dried extract of Berberis,
d) adding the solution of freeze-dried extract of Berberis to the lipid phase to obtain a hot lipid phase,
e) blending the hot lipid phase and the aqueous phase at a high speed of 5000-20,000 rpm for 5-30 minutes to obtain a hot emulsion,
f) subjecting hot emulsion obtained in step e) to two to six cycles of homogenization at 500-1200 bars pressure to obtain the lipid nano-formulation which is finally refrigerated after cooling to room temperature.

In an embodiment, the lipid nano-formulation is combined with a suitable excipient to result in a gel, a hydrogel, an organogel, a syrup, a paste, a cream, a liquid wash, a facewash, a mouthwash, an oral rinse, an ointment, a liquid ampoule, a dispersion, a nasal drops/spray, an aerosol spray, a powder, an orthotic aid, a liquid oral formulation, a facemask, a film, an implant, a tablet, lozenges, capsules, suppositories, pessaries, patches and gummies.

Further, the SLNs of freeze-dried Berberis extract are in the form of a dispersion for oral, parenteral, ocular, intranasal, dental, buccal, vaginal, rectal, otic, transdermal, and topical delivery.

In an embodiment, the disclosure provides a process for preparing the lipid nano-formulation gel of freeze-dried Berberis extract comprising:
a) dispersing a polymer in water and adding a pH adjusting agent to affect gelling of polymer and to form a translucent gel,
b) preparing a dispersion of lipid nano-formulation of freeze-dried Berberis extract, and
c) adding the dispersion of lipid nano-formulation of freeze-dried Berberis extract obtained in step b) to the gel obtained in step a) to obtain a lipid nano-formulation gel of freeze-dried Berberis extract.

In another embodiment, the polymer is selected from the group consisting of Carbopol (known as carbomers), acacia, alginic acid, bentonite, carboxymethyl cellulose, ethylcellulose, gelatin, hydroxyethyl cellulose, hydroxypropyl cellulose, magnesium aluminum silicate (Veegum), methylcellulose, poloxamers (Pluronics), polyvinyl alcohol, sodium alginate, tragacanth, and xanthan gum. In a preferred embodiment of the present invention, the polymer is carbopol or also known as carbomers.

In yet another embodiment, the pH adjusting agent is an acidifier or an alkalinizing agent. In one embodiment, the acidifier is selected from the group consisting of fumaric acid, citric acid, malic acid, tartaric acid, glycolic acid, dilute mineral acids. In another embodiment, the alkalinizing agent is selected from the group consisting of dilute solutions of sodium citrate, sodium acetate, sodium carbonate, triethanolamine, and sodium hydroxide. In one preferred embodiment, triethanolamine is used specifically for adjusting pH and stabilizing the gels and other skin care formulations.

In one embodiment, the lipid nano-formulation gel releases 95% of berberine (major active component) in 192 hours when placed in ethanol: phosphate buffer pH 6.8 (40:60) as release medium 37° C.

Accordingly, the disclosure provides a process for preparing freeze-dried Berberis extract comprising:
i. cutting and crushing plant parts to obtain a moderately fine powder of Berberis,
ii. packing the powder obtained in step i. in a polyethylene bag and storing in a freezer at −20° C. to obtain frozen Berberis powder,
iii. carrying out reflux extraction of the frozen Berberis powder with an extraction solvent at 50-90° C. for 30 minutes to 8 hours,
iv. filtering the extracts through Whatman filter paper and concentrating the extract to dryness,
v. standardizing the extract by analysing the marker content of berberine, palmatine, jatrorrhizine and magnoflorine, producing fingerprint profiles and ATR fingerprints to obtain a standardized extract of Berberis,
vi. pre-treating the standardized extract by concentrating the extract at a temperature in a range of 19° C. to 70° C. and pressure in a range of 10 mmHg/torr to 30 mmHg/torr for reducing extract volume to about 1 to 25 mL to obtain concentrated extract,
vii. freezing the extract obtained in step vi. at a temperature in a range of −30 to −50° C. for 8 to 24 hours with 1-10 mm thickness of concentrated extract,
viii. primary drying the extract obtained in step vii. at a temperature in a range of −45 to −50° C. and pressure in a range of 25 to 30 mTorr for 15 to 20 hours, and
ix. secondary drying the extract obtained in step viii. at a temperature in a range of −45 to −60° C. and pressure in a range of 25 to 30 mTorr for 15 to 48 hours to obtain the freeze-dried Berberis extract.

In a preferred embodiment, the plant part used in step i. is a stem, root, or a mixture thereof. In another preferred embodiment, the plant part used in step i. is from one of the species of genus Berberis or Mahonia.

In a preferred embodiment, the extraction of step iii. is a reflux extraction and is carried out 80° C. for 150 minutes.

In a further embodiment, the extraction solvent of step iii. is an organic or inorganic solvent.

In one embodiment, the extraction solvent of step iii. is an alcohol, water or a mixture of alcohol and water.

In a preferred embodiment, the extraction solvent is ethanol and the extraction of step iii. is performed three times with 60% ethanol, 80% ethanol and 80% ethanol at pH 1.9 maintained with 10% HCl solution.

In yet another embodiment, the optimized extraction and freeze-drying procedure increases the solubility of the Berberis extract into the surfactants and thereby increasing its drug loading and entrapment into a nano-carrier system/nano-formulation.

In a preferred embodiment, the surfactant is polysorbate 80 and solubilizing agent is polyethylene glycol (PEG 400). Coating with PEG, which is a polymer of hydrophilic nature, gave better results, since it has high hydrophilicity, chain flexibility, electrical neutrality and lacks functional groups, thereby preventing it from interacting unnecessarily with the biological components, which is very important for a suitable dosage formulation.

In a preferred embodiment, the cosurfactant used in accordance with the disclosure can be anionic, cationic, non-ionic or zwitterionic which include but is not limited to soy lecithin, egg lecithin, phosphatidylcholine; ethylene oxide copolymers, propylene oxide copolymers, poloxamers, sorbitan ethylene oxide/propylene oxide copolymers, polysorbate 20, polysorbate 60, polysorbate 80, sorbitan esters, span 20, span 40, span 60, span 80, alkyl aryl polyether alcohol polymers, tyloxapol, bile salts, cholate, glycocholate, taurocholate, taurodeoxycholate, gemini surfactants, and alcohols. The cosurfactant can also referred to as an emulsifier.

In another preferred embodiment, the disclosure also relates to the process of preparation of freeze-dried Berberis extract loaded lipid nano-formulation by hot high-pressure homogenization and assigning a lipophilic envelope to it.

In yet another embodiment, the high drug loading and efficient entrapment of the freeze-dried Berberis extract within the lipid core of these nanoparticles in a solubilized form provides protection to the extract against oxidation, hydrolytic and photo degradation, provides controlled release with increased bioavailability and thereby increases stability, efficacy, safety and entrapment.

In a further embodiment, the lipid nano-formulation of freeze-dried Berberis extract shows enhanced penetration through biological membrane due to resemblance of lipid/phospholipid to biological membranes.

In another embodiment, the lipid nano-formulation provides improved oral bioavailability or effective delivery of the freeze-dried Berberis extract to epidermal, dermal, and subcutaneous layers following topical application with enhanced drug delivery.

In a further embodiment, the freeze-dried Berberis extract loaded lipid nano-formulation is incorporated into semisolid, gel base, liquid solutions, liquid or aerosolized spray or film to increase stability and provide flexibility for the freeze-dried Berberis extract loaded lipid nano-formulation gel to be used for topical application or to be used as a liquid oral formulation or spray dried for oral administration or nasal administration or to be developed into lozenges or in cosmetics or to be used for oral, parenteral, dental, buccal, ocular, intranasal, vaginal, rectal, otic, transdermal, and topical delivery.

In yet another aspect, the formulation is used for treating diabetes and more specifically diabetes associated complications.

In yet another aspect, the formulation is used as a wound healer, an anti-infective, as an anti-inflammatory agent and as an antioxidant.

In a preferred aspect, the diabetes associated complications are one or more of cardiovascular complications, diabetic retinopathy, diabetic nephropathy, diabetic polyneuropathies, thyroid disorders, skin conditions, hearing impairment, foot complications such as leg ulcers and diabetic foot, diabetic wounds, liver diseases and reduced immunity.

In yet another embodiment, the diabetic wound is an acute or chronic wound.

In an embodiment, the percent healing of wounds in animals treated with freeze dried Berberis extract lipid nano-formulation gel is 97%.

In yet another embodiment, SLNs exhibit any therapeutic property as shown by Berberis extract.

The stem and root samples of Berberis (B. lycium) were collected from Dr. Y. S. Parmar University of Horticulture and Forestry, Nauni, District Solan (H.P.) in the month of June 2014. The identity of plant samples was established on the basis of taxonomic characters and have been authenticated by NISCAIR vide reference no. 3283-84-2. All the samples have been deposited at the museum-cum-Herbarium of University Institute of Pharmaceutical Sciences.

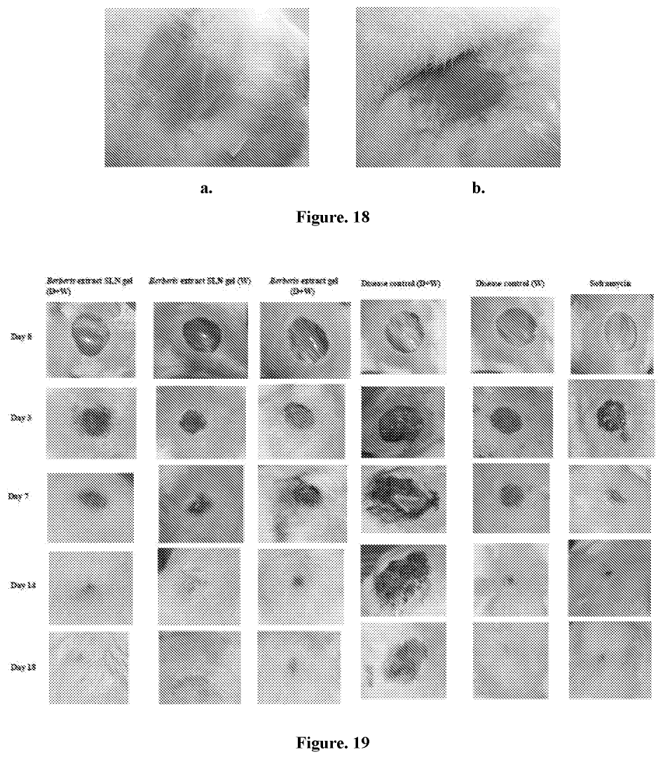

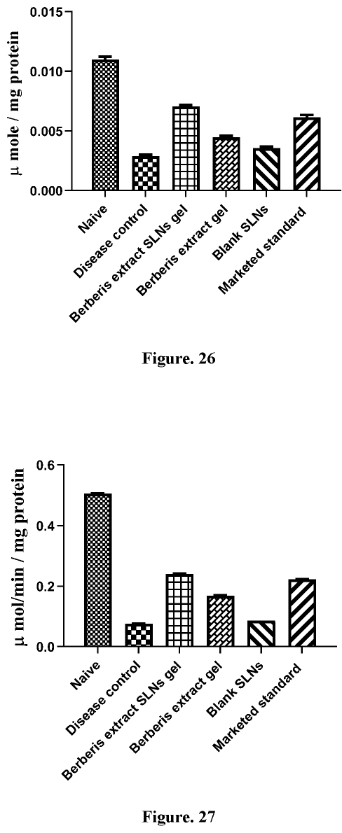

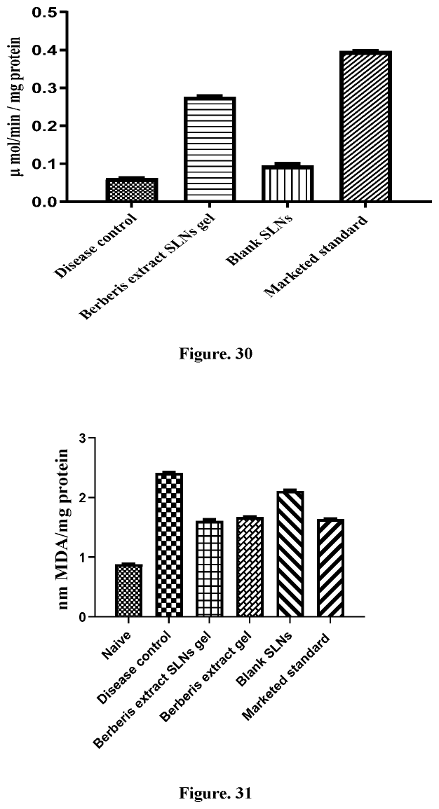

The term “Berberis extract SLNs gel” used in FIGS. 19-32 is synonymous with “freeze-dried Berberis extract SLNs gel”.

### Advantages of the Disclosed Formulation and Process:

The process and the nano-formulation system disclosed have the following characteristics which help overcome the drawbacks associated with already available formulations.
1) The lipid nano-formulation developed is of a single plant extract.
2) Standardized Berberis stem extract with 7-fold increase in its loading was prepared using optimized extraction and freeze-drying process. The optimized extraction and freeze-drying procedure increase the solubility of the Berberis extract into the surfactants enabling its incorporation into a nano-formulation system. The increase in the extract solubility increased drug loading which in turn increased drug delivery and therapeutic efficacy for faster healing of wounds.
3) The nano-formulation system is prepared using high-pressure homogenization method which is industrially viable and environment friendly as it does not make use of any organic solvents.
4) The disclosure involves preparation of aqueous dispersion of lipid nano-formulation which is easy to use.
5) The developed lipid nano-formulation have high drug loading with respect to the lipid phase, significant entrapment and smaller size. The high drug loading and efficient entrapment of the Berberis extract within the lipid core of the nano-formulation in a solubilized form provide protection to the extract against oxidation, hydrolytic and photo degradation, provide controlled release with increased bioavailability and thereby increase efficacy, stability, safety and entrapment.
6) Biocompatible, cheap, easily available GRAS components have been used.

### EXAMPLES

The disclosure will now be illustrated with working examples, which is intended to illustrate the working of disclosure and not intended to take restrictively to imply any limitations on the scope of the disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods, the exemplary methods, devices and materials are described herein. It is to be understood that this disclosure is not limited to particular methods, and experimental conditions described, as such methods and conditions may vary.

### Example 1

### Preparation of Berberis Extract

The extraction conditions were optimized for ethanol ratio, pH and extraction period using the software Design-Expert. These optimized extraction conditions were used for the preparation of the extract.

An initial step was performed to identify the active factors influencing the extraction and the magnitude of their impact. The effect of different methods of extraction, ethanol concentration, extraction time, pH and the solute/solvent ratio were studied using a single factor test to determine the influential range of these variables for interactive and in-depth investigation. The response was measured for each one factor keeping all other factors constant.

The extraction technique was selected amongst four different techniques i.e. maceration, reflux, sonication and Soxhlet. The best technique was selected on the basis of response which comprised of total bioactive alkaloids and individual berberine and palmatine content in the given residue. The residue weight obtained was maximum in Soxhlet extraction and minimum in extraction using maceration, whereas the total alkaloids and also separately berberine and palmatine alkaloids were maximum in reflux extraction and minimum in extraction using maceration. Reflux extraction provided the added advantage of temperature control which in case of Soxhlet is fixed at the boiling temperature of the solvent making the process more energy intensive. The effect of duration of extraction was studied in reflux and ultrasonication by carrying out the extraction for different time intervals. The two techniques were zeroed in on the basis of extracted amounts of alkaloids, time of extraction, energy requirements and overall economic feasibility of the extraction procedure. Reflux extraction was carried out for time intervals of 1 to 8 hours and in ultrasonicator from 5 to 45 minutes. The reflux extractions showed that the maximum residue weight was obtained with 2-hour extraction. The ultrasonic-assisted extraction showed a continuous increase in the residue weight and content of berberine from 5 to 45 min. The reflux extraction of 2 hours was inferred to be a better method of extraction than ultrasonic assisted extraction based on the residue, alkaloidal yield, scale up feasibility and industrial suitability (Gong X, Zhang Y, Pan J, Qu H (2014) Optimization of the ethanol recycling reflux extraction process for saponins using a design space approach. PLoS ONE 9(12): e114300) Further occasional but known deleterious effect of ultrasound energy (more than 20 kHz) on bioactive constituents is known. (Handa S S, Khanuja S P S, Longo G, Rakesh D D (2008) Extraction Technologies for Medicinal and Aromatic Plants, (1^{st} edn), no. 66. Italy: United Nations Industrial Development Organization and the International Centre for Science and High Technology).

The reflux extraction was performed using different concentrations of distilled ethanol (0, 20, 40, 60, 80, and 100) to study the role of alcohol concentration. The residue weight increased with increase in alcohol concentration reaching a maximum at 60% concentration and then showing a sharp decline. The berberine extraction followed a different pattern of continuous increase with the increase in the ethanol concentration. The Berberis alkaloids are known to have greater solubility in ethanol than in water which explains greater extraction of the alkaloids with increasing concentration of ethanol off-setting the loss in residue weight with increasing alcohol concentrations. (Sut S, Faggian M, Baldan V, Poloniato G, Castagliuolo I, Grabnar I, Perissutti B, Brun P, Maggi F, Voinovich D, Peron G, Dall'Acqua S. Natural deep eutectic solvents (NADES) to enhance berberine absorption: An in vivo pharmacokinetic study. Molecules. 2017; 22(11): 1921).

The effect of solvent volume on extraction of berberine was studied by varying the solute-solvent ratio from 1:12.5 to 1:100. The response clearly indicated that with the increase in the solute-solvent ratio the quantity of the extract and berberine increased. The better solute-solvent interaction and the shifted equilibrium favoured more extraction of residue and alkaloids.

Berberine and other alkaloids of Berberis are quaternary compounds with pKa of 2.47. Therefore, pH of the extraction medium was expected to impact the extraction which was checked by varying pH of the extraction solution from 1 to 5. The role of pH on extraction was studied by varying the pH of the extraction solvent from 1 to 5. The pH was observed to be a critical factor in the extraction of alkaloids (Johansen, K. T., Ebild, S. J., Christensen, S. B., Godejohann, M., Jaroszewski, J. W., 2012. Alkaloid analysis by high-performance liquid chromatography-solid phase extraction-nuclear magnetic resonance: New strategies going beyond the standard. J. Chromatogr. A 1270, 171-177). The content of berberine was maximal under strongly acidic conditions and decreased continuously to pH-4 and then reaching steady levels between pH 4 and 5.

The effect of three independent variables of i) ethanol concentration (0-100%), ii) extraction time (30-180 min) and iii) pH (1 to 3) were investigated for the five responses of a) total residue weight, b) total alkaloidal content, c) berberine content, d) palmatine content and e) antioxidant potency. Central Composite Rotatable Design (CCRD) for three variables at five levels generated twenty runs (eight factorial points, six axial points and six centre points). Each run was carried out in triplicate.

The optimal conditions for extraction of bioactive alkaloids were predicted through Design-Expert 9.0.0 software. The values of independent variables and the predicted responses are presented as follows: extraction solvent 80% aq. ethanol, the pH of extraction solvent 1.9 and extraction period of 150 min.

### Process of Extraction

The extracts were prepared using the stem part of the shrub Berberis lycium. The extracts were prepared using 2 g of moderately fine Berberis (B. lycium) stem powder passed through sieve of pore size 180 μm. The plant material was cut into slices and then crushed in an electric grinder to get the moderately fine powder (powder particles passed through sieve of nominal aperture of 355 μm and not more than 40% passed through sieve of nominal aperture of 180 μm). The powdered samples were packed in polyethylene bags and stored in freezer at −20° C. for further experiments. The extraction was carried out by reflux extraction in a 250 mL round-bottom flask. Three extractions were performed (i) 60%, and (ii) 80% ethanol concentration and (iii) 80% ethanol at pH 1.9 maintained with 10% HCl solution. The extraction was carried out at 80° C. for 150 minutes. After extraction, the extracts were filtered through Whatman filter paper and the marc was washed with 50 mL of fresh and hot extraction solvent. The extracts were concentrated to dryness by using rotary vacuum evaporator and kept in vacuum desiccator. The standardization of the extracts was done by analysing the content of the four principal markers viz. berberine, palmatine, jatrorrhizine and magnoflorine and also by analysing the fingerprint profiles using the software UNSCRAMBLER. The ATR fingerprints were also analysed for pattern recognition to establish the complete standardization protocol. The standardized extract was used for the preparation of the formulation ensuring consistency in the raw material.

### Example 2

### Process of Freeze-Drying the Berberis Extract:

The pre-treatment of the Berberis extract i.e., concentration of the extract was carried out at a temperature of 55° C. and pressure was reduced from 100 mmHg/torr to 20 mmHg/torr, to reduce extract volume to around 10 mL. Freeze drying process for drying of Berberis extract depends upon unique characteristics of the extract, its volume, and container used. Freezing of the concentrated extract obtained was done at a temperature of −40° C. for 24 hours with 5 mm thickness of extract. The process was done in petri plate of 10 cm diameter, and 2 cm height containing 5 mm thickness of sample. The primary drying of the extract was done at −46° C. temperature and 29.5 m Torr/0.039330 Bar vapour pressure for 14 hours, further the secondary drying of the sample was done at −50° C. temperature, 29.5 m Torr/0.039330 Bar vapour pressure for a duration of 18 hours to get a completely dried powder.

### Solubility Studies of Berberis Extract

Solubility of Berberis extract (80% ethanol, pH 1.9) was performed by dissolving dried extract in polyethylene glycol by increasing the amount of extract to find out its end point, by maintaining different pH i.e. acidic (4), basic (9), neutral (7). Further to evaluate the effect of drying procedure on solubility of vacuum oven-dried and freeze dried Berberis extract, solubility studies were performed similarly by increasing the amount of extract to reach its end point or saturation point. The drying conditions were optimized to achieve the maximum solubilization. The solubility of the freeze-dried extract (obtained under optimized conditions of freeze drying) in hydrophilic solvents was exceptionally good and the solubility of the extract is increased by 20 times compared to conventional vacuum oven-dried extract. The freeze-dried extract was used further in the formulation development.

### Characterization of Vacuum-Oven and Freeze-Dried Berberis Extracts

The characterization of the vacuum oven-dried and freeze-dried extract using methods such as IR, DSC, HSM, P-XRD and TGA revealed the differences in the nature of the two extracts processed by two different methods. DSC thermogram along with TGA showed vacuum-dried sample gives broad as well as sharp peaks indicating melting process and this event is also accompanied by weight loss of 70%. However, in freeze-dried sample only broad peaks are observed which depict decomposition process, whereas, in vacuum oven-dried sample decomposition is also accompanied by melting process of crystalline form. This is well supported by HSM results. The semi-crystalline nature of vacuum oven-dried sample is also supported by P-XRD as few sharp peaks depict crystalline nature of the constituents. These peaks are not present in freeze-dried sample. This shows that freeze-dried sample is more amorphous than vacuum oven-dried sample. The solubility of the freeze-dried extract (using optimized conditions of freeze drying) in hydrophilic solvents/surfactants increased by more than 20-folds compared to vacuum oven-dried extract. Therefore, enhancement in solubility of freeze-dried extract was achieved by changing its physical form from semi-crystalline to amorphous.

### Example 3

### Development of Nanoformulation of Freeze-Dried Berberis Extract

Solubility of freeze-dried Berberis extract in different solvents Freeze-dried Berberis extract was tested for its solubility in ethanol, polysorbate 20, polysorbate 60, polysorbate 80, span 20, span 40, span 60, span 80, polyethylene glycol 400, polyethylene glycol 800 in various ratios.

### Vesicular Systems

Freeze-dried Berberis extract loaded SLNs were prepared by solvent injection method using ethanol as organic solvent. In one beaker, phospholipid (span) and freeze-dried Berberis extract were dissolved in ethanol in a definite ratio and warmed to 70° C. to obtain a lipophilic phase. On the other side, PEG 400 and water were dissolved in a definite ratio to prepare aqueous phase. The aqueous mixture was kept for stirring and the temperature was maintained at 70° C. The lipophilic phase was added dropwise with stirring to the pre warmed aqueous solution with the help of pipette. The obtained mixture was kept for cooling at room temperature. After the mixture reached normal temperature, it was refrigerated.

TABLE 1
Vesicular systems tried for freeze-dried Berberis extract

| | Lipophilic phase | | | | Hydrophilic phase | |
|---|---|---|---|---|---|---|
| Extract | Ethanol | Span 40 | Span 60 | Span 80 | PEG 400 | Water |
| Ethanol:water | (mL) | (mg) | (mg) | (mg) | (g) | (mL) |
| 60:40 | 10 | 80 | — | — | 1.2 | 8.8 |
| 60:40 | 10 | 80 | — | — | 1 | 9 |
| 60:40 | 10 | — | 40 | 40 | 1 | 9 |
| 100:0 | 10 | — | 40 | 40 | 2 | 8 |
| 100:0 | 10 | — | 40 | 40 | 0.5 | 9.5 |

### Example 4

### Preparation of Berberis Extract Loaded Solid Lipid Nanoparticles by High Pressure Homogenization

The formulation of nanoparticles was prepared by using hot homogenization technique. Soy lecithin (7 g) and water (45.7 mL) were taken together in a beaker and heated to the lipid melting temperature i.e. 82° C. to obtain an aqueous phase. The lipid, Compitrol (2.8 g) was also melted at 82° C. separately to obtain a lipid phase Berberis extract (500 mg) was dissolved in PEG 400 (7 g). This dissolved extract of Berberis was then added to the lipid phase. The hot lipid phase was once dropped into the aqueous phase under speed homogenization at 8000 rpm for 15 minutes to obtain a hot emulsion. The hot emulsion thus formed was passed through homogenizer at 1000 bars pressure and three cycles were run. The formulation so formed was cooled to room temperature, for an hour, to allow the lipidic particles to solidify after which it was refrigerated.

Incorporation of Berberis Extract Loaded SLNs into a Gel System

For enhancing the acceptability and increasing the patient compliance, the prepared Berberis extract loaded SLNs were incorporated into a hydrogel base. The latter is purported to improve the spreadability of the prepared SLN dispersion on topical application.

Hydrogel base: Carbopol (2.5% w/v) (1.8 g) was dispersed in 10.2 mL of water and kept overnight for swelling. Triethanolamine around 2-3 drops was added to this mixture with continuous stirring, to effect gelling of carbopol. Stirring was continued until a translucent gel was formed. Then, 140 mL of Berberis loaded SLN dispersion was added to the prepared gel and mixed slowly to obtain a homogenous mixture containing carbopol and 7.14 mg/mL concentration of Berberis extract. Free drug gel was prepared by incorporation of Berberis extract (7.1 mg) in a similar way as for incorporation of Berberis extract loaded SLNs.

### Example 5

### Preparation of Freeze-Dried Berberis Extract Loaded Solid Lipid Nanoparticles (SLNs) by High Pressure Homogenization Method:

The formulation of nanoparticles was prepared by using hot homogenization technique. Phospholipon 90 G (0.4 g), tween 80 (8 g) and water (78.6 mL) were taken together in a beaker and heated to the lipid melting temperature to obtain an aqueous phase. The lipid, stearic acid (4 g) was melted separately at 80° C. to obtain a lipid phase. Freeze-dried Berberis extract (1 g) was dissolved in PEG 400 (8 g). This dissolved freeze-dried extract of Berberis was then added to the lipid phase. The hot lipid phase was once dropped into the aqueous phase under speed homogenization at 10000 rpm for 10 minutes to obtain a hot emulsion. The hot emulsion thus formed was passed through homogenizer at 1200 bars pressure and two cycles were run. The formulation so formed was cooled to room temperature, for an hour, to allow lipidic particles to solidify after which it was refrigerated.

Incorporation of SLNs into Gel System

Carbopol 2 g was dispersed in 10 mL of water and kept overnight for swelling. 2-3 drops of triethanolamine was added drop wise to this mixture with continuous stirring, to affect the gelling of carbopol and maintain the pH. Stirring was continued until a translucent gel was formed. Then 88 mL of freeze-dried Berberis extract loaded SLNs dispersion was added to the prepared gel and mixed slowly to obtain a homogeneous mixture containing carbopol. Similarly, a Blank SLNs gel was prepared by dispersing freeze-dried Berberis extract free SLNs in 88.5 mL of double distilled water.

### Example 6

### Preparation of Freeze-Dried Berberis Extract Loaded Solid Lipid Nanoparticles (SLNs) by High Pressure Homogenization Method:

The formulation of nanoparticles was prepared by using hot homogenization technique. Phospholipon 90 G (0.4 g), tween 80 (8 g) and water (67.6 mL) were taken together in a beaker and heated to the lipid melting temperature to obtain an aqueous phase. The lipid, Precirol (4 g) was melted separately at 70° C. to obtain a lipid phase. Freeze-dried Berberis extract (10 g) was dissolved in PEG 400 (10 g). This dissolved freeze-dried extract of Berberis was then added to the lipid phase. The hot lipid phase was once dropped into the aqueous phase under speed homogenization at 10000 rpm for 15 minutes to obtain a hot emulsion. The hot emulsion thus formed was passed through homogenizer at 500 bars pressure and six cycles were run. The formulation so formed was cooled to room temperature, for an hour, to allow lipidic particles to solidify after which it was refrigerated.

Incorporation of SLNs into Gel System

Carbopol 1.5 g was dispersed in 10 mL of water and kept overnight for swelling. 2-3 drops of triethanolamine was added drop wise to this mixture with continuous stirring, to affect the gelling of carbopol and maintain the pH. Then 88.5 mL of freeze-dried Berberis extract loaded SLNs dispersion was added to the prepared gel and mixed slowly to obtain a homogeneous mixture containing carbopol. Similarly, a Blank SLNs gel was prepared by dispersing freeze-dried Berberis extract free SLNs in 88.5 mL of double distilled water.

### Example 7

### Preparation of Freeze-Dried Berberis Extract Loaded Solid Lipid Nanoparticles (SLNs) by High Pressure Homogenization Method:

The formulation of nanoparticles was prepared by using hot homogenization technique. Phospholipon 90 G (0.4 g), tween 80 (7 g) and water (78.6 mL) were taken together in a beaker and heated to the lipid melting temperature to obtain an aqueous phase. The lipid, Glycerol monostearate (4 g) was melted separately at 85° C. to obtain a lipid phase. Freeze-dried Berberis extract (2 g) was dissolved in PEG 400 (8 g). This dissolved freeze-dried extract of freeze-dried Berberis was then added to the lipid phase. The hot lipid phase was once dropped into the aqueous phase under speed homogenization at 12000 rpm for 12 minutes to obtain a hot emulsion. The hot emulsion thus formed was passed through homogenizer at 800 bars pressure and five cycles were run. The formulation so formed was cooled to room temperature, for an hour, to allow lipidic particles to solidify after which it was refrigerated.

Incorporation of SLNs into Gel System

Carbopol 0.5 g was dispersed in 10 mL of water and kept overnight for swelling. 2-3 drops of triethanolamine was added drop wise to this mixture with continuous stirring, to affect the gelling of carbopol and maintain the pH. Stirring was continued until a translucent gel was formed. Then 89.5 mL of freeze-dried Berberis extract loaded SLNs dispersion was added to the prepared gel and mixed slowly to obtain a homogeneous mixture containing carbopol. Similarly, a Blank SLNs gel was prepared by dispersing freeze-dried Berberis extract free SLNs in 88.5 mL of double distilled water.

### Example 8

### Preparation of SLNs of Freeze-Dried Berberis Extract

Freeze-dried Berberis extract loaded nanoparticles were prepared using high pressure homogenization technique. Compritol® 888 ATO was chosen as the lipid component as it has more ability to solubilize than other lipids screened such as stearic acid, glycerol monostearate, Precirol® ATO 5, tripalmitin (C16) and trilaurin (C12). Polyethylene glycol (PEG) was used as a surfactant because the freeze-dried Berberis extract dissolved well in it. PEG, a polymer of hydrophilic nature showed better results as it has high hydrophilicity, chain flexibility, electrical neutrality and absence of functional groups, preventing it from interacting unnecessarily with the biological components, an important aspect of a good formulation.

### Preparation of Freeze-Dried Berberis Extract Loaded Solid Lipid Nanoparticles (SLNs) by High Pressure Homogenization Method:

The formulation of nanoparticles was prepared by using hot homogenization technique. Phospholipon 90 G (0.4 g), tween 80 (8 g) and water (78.6 mL) were taken together in a beaker and heated to the lipid melting temperature to obtain an aqueous phase. The lipid, Compritol® 888 ATO (4 g) was melted separately at 82° C. to obtain a lipid phase. Freeze-dried Berberis extract (0.5 g) was dissolved in PEG 400 (8.5 g). This dissolved freeze-dried extract of Berberis was then added to the lipid phase. The hot lipid phase was once dropped into the aqueous phase under speed homogenization at 8000 rpm for 15 minutes to obtain a hot emulsion. The hot emulsion thus formed was passed through homogenizer at 900 bars pressure and four cycles were run. The formulation so formed was cooled to room temperature, for an hour, to allow lipidic particles to solidify after which it was refrigerated.

Incorporation of SLNs into Gel System

Carbopol 1.5 g was dispersed in 10 mL of water and kept overnight for swelling. 2-3 drops of triethanolamine was added drop wise to this mixture with continuous stirring, to affect the gelling of carbopol and maintain the pH. Stirring was continued until a translucent gel was formed. Then 88.5 mL of freeze-dried Berberis extract loaded SLNs dispersion was added to the prepared gel and mixed slowly to obtain a homogeneous mixture containing carbopol. Similarly, a Blank SLNs gel was prepared by dispersing freeze-dried Berberis extract free SLNs in 88.5 mL of double distilled water.

### Example 9

### Preparation of Freeze-Dried Berberis Extract Loaded Solid Lipid Nanoparticles (SLNs) by High Pressure Homogenization Method:

The formulation of nanoparticles was prepared by using hot homogenization technique. Phospholipon 90 G (0.7 g), tween 80 (9 g) and water (71.3 mL) were taken together in a beaker and heated to the lipid melting temperature to obtain an aqueous phase. The lipid, Compritol® 888 ATO (4 g) was melted separately at 82° C. to obtain a lipid phase. Freeze-dried Berberis extract (5 g) was dissolved in PEG 400 (10 g). This dissolved freeze-dried extract of Berberis was then added to the lipid phase. The hot lipid phase was once dropped into the aqueous phase under speed homogenization at 8000 rpm for 20 minutes to obtain a hot emulsion. The hot emulsion thus formed was passed through homogenizer at 1000 bars pressure and three cycles were run. The formulation so formed was cooled to room temperature, for an hour, to allow lipidic particles to solidify after which it was refrigerated.

Incorporation of SLNs into Gel System

Carbopol 1.5 g was dispersed in 10 mL of water and kept overnight for swelling. 2-3 drops of triethanolamine was added drop wise to this mixture with continuous stirring, to affect the gelling of carbopol and maintain the pH. Stirring was continued until a translucent gel was formed. Then 88.5 mL of freeze-dried Berberis extract loaded SLNs dispersion was added to the prepared gel and mixed slowly to obtain a homogeneous mixture containing carbopol. Similarly, a Blank SLNs gel was prepared by dispersing freeze-dried Berberis extract free SLNs in 88.5 mL of double distilled water.

### Example 10

### Preparation of Freeze-Dried Berberis Extract Loaded Solid Lipid Nanoparticles (SLNs) by High Pressure Homogenization Method:

The formulation of nanoparticles was prepared by using hot homogenization technique. Phospholipon 90 G (0.2 g), tween 80 (10 g) and water (76.8 mL) were taken together in a beaker and heated to the lipid melting temperature to obtain an aqueous phase. The lipid, Compritol® 888 ATO (4 g) was melted separately at 82° C. to obtain a lipid phase. Freeze-dried Berberis extract (1.8 g) was dissolved in PEG 400 (7.2 g). This dissolved freeze-dried extract of Berberis was then added to the lipid phase. The hot lipid phase was once dropped into the aqueous phase under speed homogenization at 5000 rpm for 30 minutes to obtain a hot emulsion. The hot emulsion thus formed was passed through homogenizer at 1000 bars pressure and three cycles were run. The formulation so formed was cooled to room temperature, for an hour, to allow lipidic particles to solidify after which it was refrigerated.

Incorporation of SLNs into Gel System

Carbopol 1 g was dispersed in 10 mL of water and kept overnight for swelling. 2-3 drops of triethanolamine was added drop wise to this mixture with continuous stirring, to affect the gelling of carbopol and maintain the pH. Stirring was continued until a translucent gel was formed. Then 89 mL of freeze-dried Berberis extract loaded SLNs dispersion was added to the prepared gel and mixed slowly to obtain a homogeneous mixture containing carbopol. Similarly, a Blank SLNs gel was prepared by dispersing freeze-dried Berberis extract free SLNs in 89 mL of double distilled water.

### Example 11

### Preparation of Freeze-Dried Berberis Extract Loaded Solid Lipid Nanoparticles (SLNs) by High Pressure Homogenization Method:

The formulation of nanoparticles was prepared by using hot homogenization technique. Phospholipon 90 G (0.7 g), tween 80 (9 g) and water (73.3 mL) were taken together in a beaker and heated to the lipid melting temperature to obtain an aqueous phase. The lipid, Compritol® 888 ATO (2 g) was melted separately at 82° C. to obtain a lipid phase. Freeze-dried Berberis extract (6 g) was dissolved in PEG 400 (9 g). This dissolved freeze-dried extract of Berberis was then added to the lipid phase. The hot lipid phase was once dropped into the aqueous phase under speed homogenization at 20000 rpm for 5 minutes to obtain a hot emulsion. The hot emulsion thus formed was passed through homogenizer at 1000 bars pressure and three cycles were run. The formulation so formed was cooled to room temperature, for an hour, to allow lipidic particles to solidify after which it was refrigerated.

Incorporation of SLNs into Gel System

Carbopol 2 g was dispersed in 10 mL of water and kept overnight for swelling. 2-3 drops of triethanolamine was added drop wise to this mixture with continuous stirring, to affect the gelling of carbopol and maintain the pH. Stirring was continued until a translucent gel was formed. Then 88 mL of freeze-dried Berberis extract loaded SLNs dispersion was added to the prepared gel and mixed slowly to obtain a homogeneous mixture containing carbopol. Similarly, a Blank SLNs gel was prepared by dispersing freeze-dried Berberis extract free SLNs in 88 mL of double distilled water.

### Example 12

### Preparation of Freeze-Dried Berberis Extract Loaded Solid Lipid Nanoparticles (SLNs) by High Pressure Homogenization Method:

The formulation of nanoparticles was prepared by using hot homogenization technique. Phospholipon 90 G (0.4 g), tween 80 (9 g) and water (70.6 mL) were taken together in a beaker and heated to the lipid melting temperature to obtain an aqueous phase. The lipid, Compritol® 888 ATO (2 g) was melted separately at 82° C. to obtain a lipid phase. Freeze-dried Berberis extract (8 g) was dissolved in PEG 400 (10 g). This dissolved freeze-dried extract of Berberis was then added to the lipid phase. The hot lipid phase was once dropped into the aqueous phase under speed homogenization at 15000 rpm for 10 minutes to obtain a hot emulsion. The hot emulsion thus formed was passed through homogenizer at 1000 bars pressure and three cycles were run. The formulation so formed was cooled to room temperature, for an hour, to allow lipidic particles to solidify after which it was refrigerated.

Incorporation of SLNs into Gel System

Carbopol 2 g was dispersed in 10 mL of water and kept overnight for swelling. 2-3 drops of triethanolamine was added drop wise to this mixture with continuous stirring, to affect the gelling of carbopol and maintain the pH. Stirring was continued until a translucent gel was formed. Then 88 mL of freeze-dried Berberis extract loaded SLNs dispersion was added to the prepared gel and mixed slowly to obtain a homogeneous mixture containing carbopol. Similarly, a Blank SLNs gel was prepared by dispersing freeze-dried Berberis extract free SLNs in 88 mL of double distilled water.

### Example 13

### Characterization of SLNs

### Optical Microscopy

SLNs of freeze-dried Berberis extract were characterized preliminarily using optical microscope after suitable dilution with distilled water.

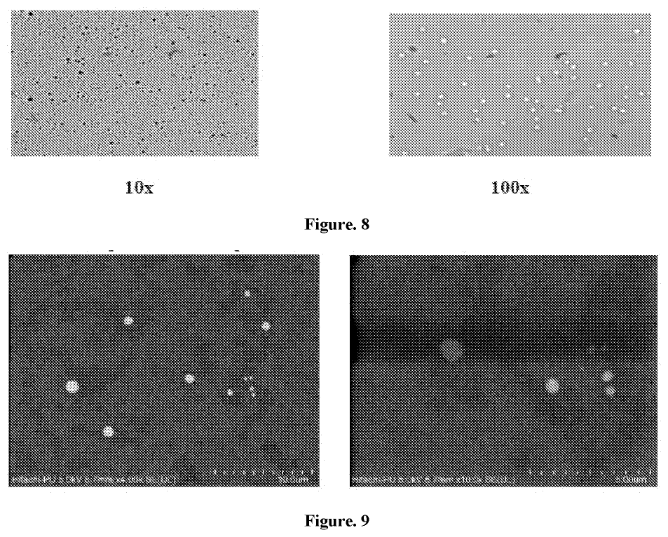

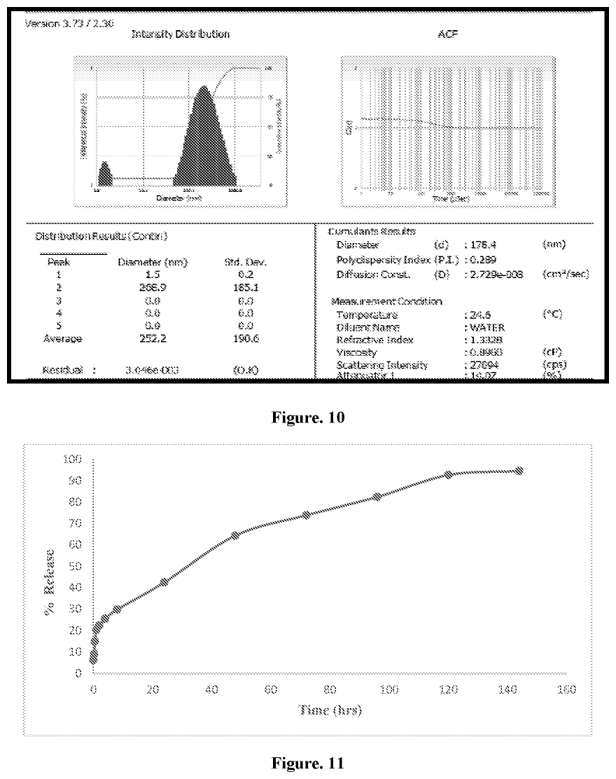

Result: The general optical inspection of freeze-dried Berberis extract SLNs indicated these to be small and round in shape, with no aggregation/irregularities as shown in FIG. 8.

### Field Emission Scanning Electron Microscopy (FESEM)

SLNs dispersion of freeze-dried Berberis extract was placed on Nucleopore Track-Etch membrane and kept for drying at room temperature. Further, silicon wafer was attached to the dried membrane followed by sputter coating with platinum. Images were taken at the −140° C. and a voltage of 15 kV using field emission scanning electron microscope (FESEM).

Result: FESEM of SLNs of freeze-dried Berberis extract depicted particles were spherical in shape and are under nanoscale range as shown in FIG. 9.

### Particle Size

Diameter of SLNs in a dispersion (10×) dilution was determined using laser diffraction. Result: The developed freeze-dried Berberis extract SLNs showed an average particle size of 178.4 nm with an average polydispersity index (PDI) of 0.289. After 8 months, freeze-dried Berberis extract SLNs showed particle size of 166.1 nm with PDI of 0.353 which showed that the developed SLNs system was stable.

### Assay/Total Drug Content (TDC)

The TDC of SLNs was determined by disrupting 1 mL of the dispersion using mixture of chloroform-methanol (1:1) using vortex and the solution thus obtained was filtered through Whatman filter paper to obtain a clear solution. The obtained solutions were analysed spectrophotometrically at λₘₐₓ 345 nm for freeze-dried Berberis extract SLN formulation using chloroform-methanol (1:1) as blank. TDC was calculated by using the following equation:

$$Total drug content ( mg ) = \frac{Absorbance * 10 * Dilution factor}{\text{?}}$$

$$Total drug content ( \% ) = \frac{Actual drug content ( mg )}{Theortical drug content ( mg )} \times 100$$

$$\text{?} \text{indicates text missing or illegible when filed}$$

Result: Assay/total drug content of developed freeze-dried Berberis extract SLNs was 86.23±1.20%. High values (approaching 100%) of TDC are indicative of the insignificant loss incurred during the process of preparation of SLNs.

### Determination of Entrapment Efficiency (EE)

The EE of the prepared SLNs was determined by using dialysis membrane having pore size 2.4 nm, molecular weight cut off 12-14 KD. Membrane was soaked in double distilled water for 12 hours prior to use. For freeze-dried Berberis extract loaded SLNs dispersion, 1 mL was taken and diluted to 10 mL and 0.5 mL of diluted SLNs was placed in pre-soaked dialysis tubing, which was hermetically sealed, and dialyzed against methanol 100 mL at room temperature for 2 hours. The amount of drug released into receptor/release medium was analysed spectrophotometrically with appropriate dilution. The SLNs retained in the dialysis bag were disrupted by appropriate dilution with a mixture of chloroform and methanol (1:1) to calculate the amount of drug entrapped within SLNs. Entrapment efficiency was calculated using the expression.

$$Entrapment efficiency ( \% ) = \frac{Entrapped drug content ( mg )}{Actual drug content ( mg )} \times 100$$

Result: Entrapment efficiency (EE) of freeze-dried Berberis extract SLNs was 90.56% 1.25%. Higher EE indicates the suitability of the method used for the preparation of SLNs.

### Zeta Potential

Zeta potential of SLNs dispersion (10×) dilution was measured using Beckman zetasizer, at 25° C. and the electric field strength of 23.2 V/cm, using high concentration cell. The zetasizer measures the zeta potential based on the Smoluchowski equation. The observed zeta potential of freeze-dried Berberis extract SLNs was −6.97 mV. The near neutral zeta potential has earlier been reported to result in stable particles. (Ref: Bhandari R, Kaur I P. Pharmacokinetics, tissue distribution and relative bioavailability of isoniazid-solid lipid nanoparticles. Int J Pharm. 2013; 441:202-12.)

### In Vitro Release Studies

For freeze-dried Berberis extract loaded SLNs, ethanol: phosphate buffer pH 6.8 (40:60) was taken as release medium. The dialysis membrane was loaded with 0.5 mL of SLNs dispersion. Aliquots of 0.5 mL were withdrawn at different time intervals for 120 hours and replenished with same volume of release medium. The collected samples were suitably diluted and analysed by UV-visible spectrophotometer. Corresponding amount of PEG-400 used in formulation was added to the release medium used for in-vitro release to balance the osmotic pressure on either side of the dialysis membrane. In vitro release profile can reveal fundamental information on the dosage form and its behavior. It also provides details on the release mechanism and kinetics enabling a rational and scientific approach to drug development.

Result: For freeze-dried Berberis extract SLNs, 93% release of berberine (major active marker) was observed in a time period of 120 hours as depicted in FIG. 11. The increased release span was beneficial for prolonged pharmacological activity.

### FT-IR

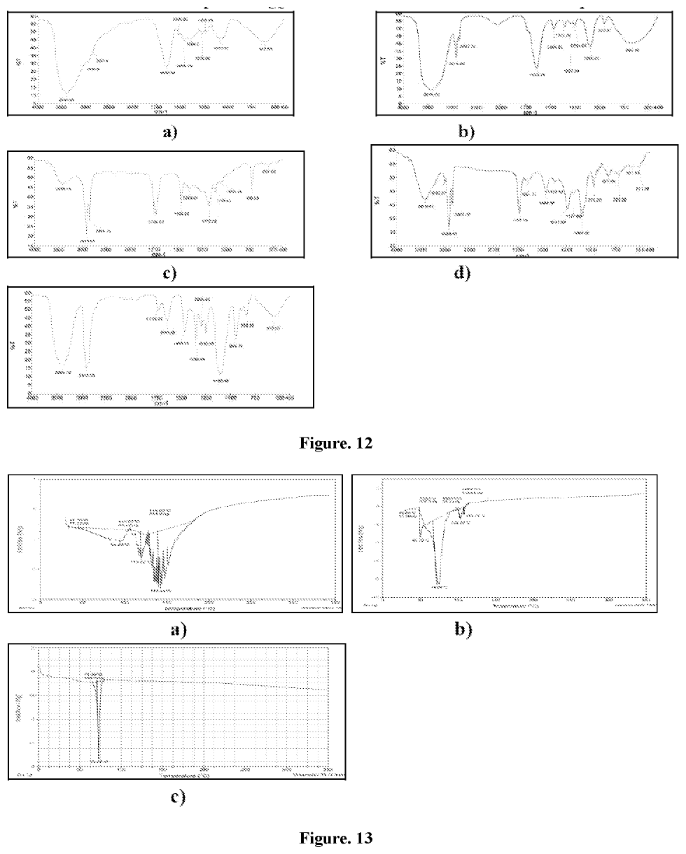

The FT-IR spectra (Agilent Technologies 630 Cary, USA) of freeze-dried Berberis extract, freeze-dried Berberis extract loaded SLNs, Compritol® 888 ATO, phospholipon 90G and physical mixture of Compritol® 888 ATO with PEG 400 melted and solidified were obtained, using Micro Lab software. Samples were analysed over the range 400-4000 cm⁻¹. Result: FT-IR spectra of freeze-dried Berberis extract, freeze-dried Berberis extract SLNs, Compritol® 888 ATO (C), Phospholipon 90 G and physical mixture of Compritol® 888 ATO and PEG 400 (melted and solidified) are shown in FIG. 12. Freeze-dried Berberis extract showed characteristic absorption bands at 1274.60 cm⁻¹ due to C—O—C asymmetrical and symmetrical stretching due to presence of ether and at 1075.57 cm⁻¹ due to C—O stretching vibrations. Compritol® 888 ATO showed peaks at 2918.62 cm⁻¹ and 2849.75 cm⁻¹ (alkane stretch) and 1738 cm⁻¹ (ethers stretch). All characteristic absorption bands of freeze-dried Berberis extract, Compritol® 888 ATO were observed in Berberis extract SLNs with observed reduction in the sharpness of some peaks. The spectra of Compritol® 888 ATO and PEG 400 melted and solidified showed no signifying interaction between the two even on heating. This confirmed that both these components retain their individual properties when incorporated in nanoparticles. The retention of characteristic peaks of individual components suggested absence of interaction of the components in the product.

### Differential Scanning Calorimetry (DSC)

DSC thermographs of freeze-dried Berberis extract, freeze-dried Berberis extract loaded SLNs, Compritol® 888 ATO were recorded to evaluate any significant shift or disappearance/appearance of new peaks. Different lipid modifications possess different melting points and enthalpies which provides information regarding sample structure and interactions between the components. The samples (2-5 mg) were scanned using crimped aluminium pans. The temperature range was varied from 30° C. to 350° C. at the heating rate of 10° C./min.

Result: DSC thermograms of freeze-dried Berberis extract, freeze-dried Berberis extract SLNs and Compritol® 888 ATO are shown in FIG. 13. DSC was performed to investigate the melting behaviour of crystalline materials. DSC thermogram of freeze-dried Berberis extract showed melting point endotherm at 119.82° C. Compritol® 888 ATO showed sharp endotherm at 72.85° C. Freeze-dried Berberis extract loaded SLNs showed large peak at 74.94° C. along with disappearance of the peaks observed in freeze-dried Berberis extract thermogram. This is indicative of the molecular dispersion of freeze-dried Berberis extract into the loaded SLNs, its existence in amorphous state and efficient entrapment in the lipid matrix.

### PXRD

PXRD was performed using XPERT-PRO diffractometer system (PANalytical, Netherlands) with a CuKα radiation (1.54060 A°) of lyophilized Berberis extract loaded SLNs, Compritol® 888 ATO, phospholipon 90G and physical mixture of Compritol® 888 ATO with PEG 400 melted and solidified. The tube voltage and current were set at 45 kV and 40 mA respectively. The divergence slit and anti-scattering slit setting were set at 0.44° for the irradiation on the 10 mm specimen length. Each sample was packed in an aluminium sample holder and measured by continuous scan from 4.0084° to 49.9934°, 2θ at a step size of 0.0170° C. and scan time of 25 s.

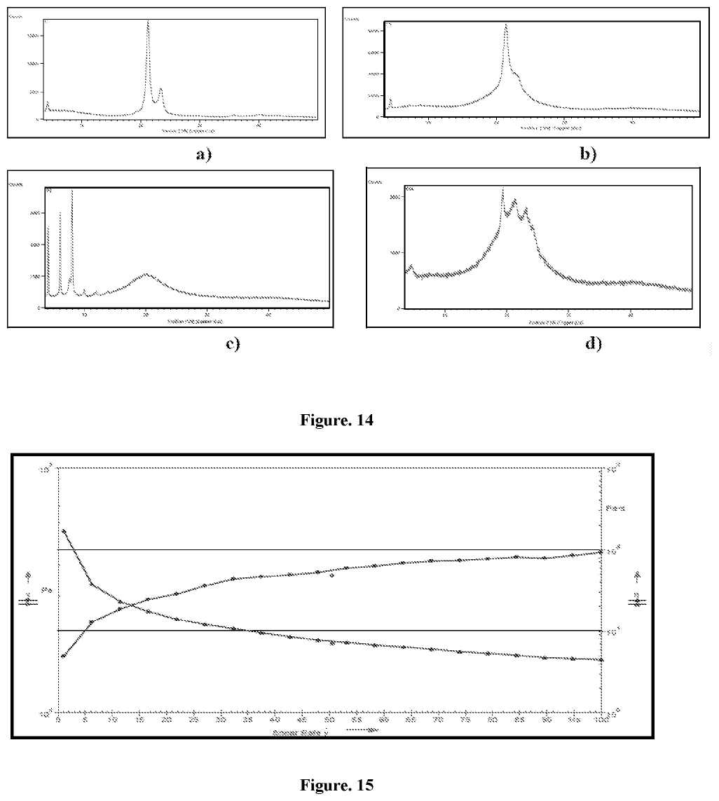

Result: PXRD of Compritol® 888 ATO, Compritol® 888 ATO melted with PEG 400, phospholipon 90 G, freeze-dried Berberis extract SLNs are shown in FIG. 14. XRD pattern of Compritol® 888 ATO showed sharp peaks at 2 scattered angles 21.19 and 23.29 indicating its crystalline state. Compritol® 888 ATO melted with PEG 400 (2θ of 21.4 and 22.8) showed pattern of peaks similar to that exhibited by Compritol® 888 ATO alone. This confirms the findings obtained from their FTIR spectra that PEG 400 does not interact with Compritol® 888 ATO. Freeze-dried Berberis extract loaded SLNs showed broad and diffused peaks with low intensities thus indicating amorphous nature in formulations.

### Example 14

### Characterization of Gels

### Rheological Studies

Rheological study of the prepared SLNs gels of freeze-dried Berberis extract was performed using a rotational type rheometer (Rheolab Q C, Anton Par GmbH, Vienna, Austria attached with a water jacket (C-LTD80/QC) for maintaining constant temperature. Data analysis was carried out using Rheoplus/32, version 3.40. ACC27 spindle geometry was used for measuring the torque as well as viscosity of the sample. Temperature was kept at 30° C. and shear rate was selected from 0 to 100 si.

Result: Rheological behavior of any formulation depicts the structural changes that occur within a system on application of shear during its processing, storage, and usage. Rheological profile of the SLNs gels and freeze-dried Berberis extract loaded SLNs gel are shown in FIG. 15. It was obtained by measuring the shear rate at different shear stress values, initially by increasing and then by decreasing the speed of rotation of the spindle of the viscometer. The viscosity of the formulation decreased with increase in shear stress suggesting a shear thinning system. Shear thinning is a desirable property of topical gels as it facilitates ease of application. The decrease in viscosity with increasing shear rate also suggested a non-flocculated system with a very small particle size pointing towards stability of the system.

pH

pH of freeze-dried Berberis extract loaded SLNs gel was measured using L1-120 pH meter (Elico, Mumbai, India).

Result: The observed pH of freeze-dried Berberis extract SLNs gel was 5.8±0.5, which is near to skin pH of 5.5.

### Texture Analysis (Spreadability)

Texture profile analysis was performed to study other rheological characteristics, i.e; firmness and stickiness, of the formulation freeze-dried Berberis extract loaded SLNs gel using TTC spreadability rig fitted on Texture Analyzer™ (M/s Stable Micro Systems Ltd; UK). About 10 g of formulation was pressed to remove any air pockets (entrapped air). Excess formulation was scraped off to leave a flat test area.

Before testing, the upper cone probe (male cone) was calibrated against lower cone so that the starting point was at the same height for each test approx. 25 mm above to the lower cone. During testing, upper conical probe approached and then penetrated into sample and continued to depth 2 mm above the sample holder surfaces i.e. probe moved a distance of 23 mm from its starting point (test speed 3 mm/sec). The force encountered by the male cone to break away from the gel when starting to ascend (the point of maximum force) was measured. The value of the peak force was taken as the measurement of gel strength; the higher the value better is the strength of gel network. The area of the curve up to this point was taken as the measurement of work of shear, reflecting the work of spreadability of the sample. The negative region of the graph, produced on probe return, was a result of the weight of the sample which is lifted primarily on the upper surface of the male cone on return. This is due to back movement and hence, provides an indication of adhesion or resistance to flow off the disc. The maximum negative value is the force of adhesion for the gel and it represents the force required to extrude gel from tube. The area of the negative region of the curve was taken as work of adhesion or stickiness.

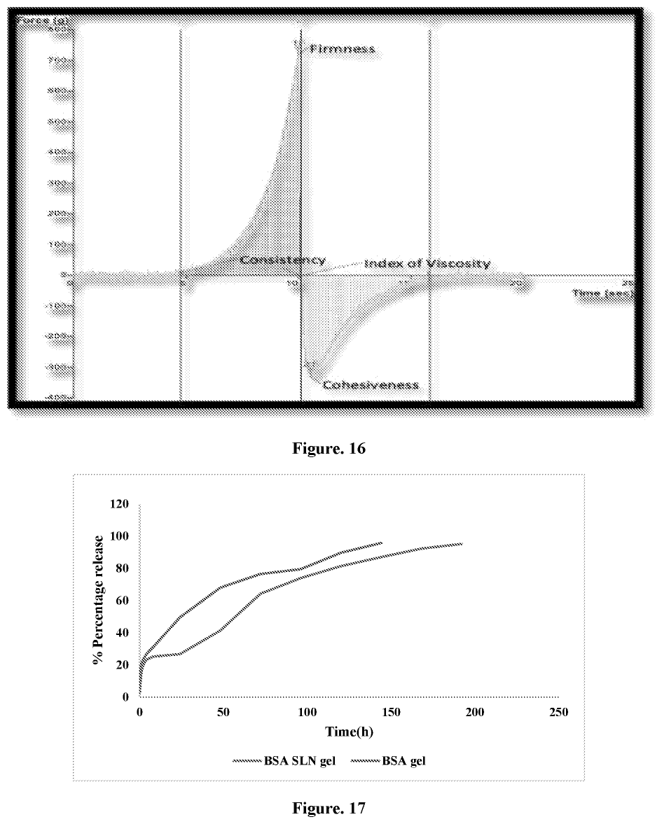

Result: Spreadability is one of the important factors for topical formulations and is responsible for stickiness, ease of application, extrudability from the container and overall consumer acceptability of a product. The results from texture analysis included firmness, consistency (work of shear), cohesiveness (extrusion force) and index of viscosity (work of adhesion) as shown in Table-2. It revealed that the developed formulations of freeze-dried Berberis extract loaded SLNs gel exhibited fairly good strength, ease of spreading and extrusion from container FIG. 16. It also possessed adequate cohesiveness, which is usually essential to hold the formulation at the site of action.

TABLE 2
Texture analysis parameters

| | | | | Index of |
|---|---|---|---|---|
| | Firmness | Consistency | Cohesiveness | viscosity |
| Formulation | (g) | (g · sec) | (g) | (g · sec) |
| Freeze-dried | 718 | 1035.54 | −330.39 | −724.09 |
| Berberis extract | | | | |
| loaded SLNs gel | | | | |

### In-Vitro Release Studies

In-vitro release studies were performed using Franz diffusion assembly. Dialysis membrane with a molecular weight cut-off of 12000-14000 Da was used after soaking in double distilled water for 12 hours. The release medium (30 mL) in each cell was maintained at 37° C. and stirred throughout the time of experiment. For freeze-dried Berberis extract loaded SLNs gel and freeze-dried Berberis extract gel in-vitro release studies ethanol: phosphate buffer pH 6.8 (40:60) was taken as release medium. The dialysis membrane was loaded with 500 mg of SLNs gel. Aliquots of 0.5 mL were withdrawn at different time intervals for 120 hours and replenished with same volume of release medium. The collected samples were suitably diluted and analysed by UV-visible spectrophotometer. For freeze-dried Berberis extract gel, dialysis membrane was loaded with 500 mg of freeze-dried Berberis extract gel instead of SLNs gel and similar procedure was followed as for freeze-dried Berberis extract loaded SLNs gel. Corresponding amount of PEG-400 used in formulations was added to the dialysate used for in-vitro release to balance the osmotic pressure on either side of the dialysis membrane.

Result: In vitro release profile of freeze-dried Berberis extract loaded SLNs gel at 37±0.5° C. is shown in FIG. 17. Freeze-dried Berberis extract loaded SLNs gel showed a prolonged release with 95% of berberine (major active marker) released in 192 hours. For comparison, freeze-dried Berberis extract gel release studies were also performed. Berberis extract gel showed 96% release of berberine (major active marker) in 144 hours. From the release studies, it can be inferred that due to formation of solid lipid nanoparticles release span of freeze-dried Berberis extract was increased which is beneficial for prolonged pharmacological activity.

### Antimicrobial Activity

The standardized B. lycium stem extract prepared using optimized extraction procedure was tested for a panel of bacterial strains including Pseudomonas aeruginosa (ATCC 27853), Acinetobacter baumannii (ATCC 19606), Staphylococcus aureus (ATCC 29213) and clinical isolates of Klebsiella pneumoniae, Staphylococcus epidermidis, Staphylococcus hemolyticus and Burkholderia cepacia complex using serial dilution method. Ceftazidime was used as the standard antibiotic for validating the protocol. The minimum inhibitory concentrations (MIC) against all seven strains are shown in Table 3.

TABLE 3
Minimum inhibitory concentrations (MIC) of Berberis
extract against different bacterial strains.

| Bacterial strain | MIC (μg/mL) |
|---|---|
| Staphylococcus aureus | 256 |
| Staphylococcus epidermidis | 64 |
| Staphylococcus hemolyticus | 512 |
| Acinetobacter baumanni | 1024 |
| Burkholderia cepacia complex | 2048 |
| Klebsiella pneumoniae | — |
| Pseudomonas aeruginosa | 4096 |

### Example 15

### Pharmacodynamic Activity in Animals

### Acute Dermal Irritation Studies as Per OECD Guidelines 404

The scores for dermal irritation study are compiled in table 4. Zero score value clearly demonstrates a non-irritant nature of the developed formulation when applied to dermal tissues, and hence is concluded to be safe for topical application. The study was conducted in accordance with OECD guidelines. FIG. 18 demonstrates the test animal before and after the application of formulation.

TABLE 4
Scores of acute dermal irritation study of freeze-dried Berberis extract
loaded SLNs in rabbits

| Tissue | Rabbit 1 | | | | | Rabbit 2 | | | | | Rabbit 3 | | | | | |
|---|---|---|---|---|---|---|---|---|---|---|---|---|---|---|---|---|
| reaction | 0 h | 1 h | 24 h | 48 h | 72 h | 0 h | 1 h | 24 h | 48 h | 72 h | 0 h | 1 h | 24 h | 48 h | 72 h | Score |
| Erythema | 0 | 0 | 0 | 0 | 0 | 0 | 0 | 0 | 0 | 0 | 0 | 0 | 0 | 0 | 0 | 0/60 |
| Oedema | 0 | 0 | 0 | 0 | 0 | 0 | 0 | 0 | 0 | 0 | 0 | 0 | 0 | 0 | 0 | 0/60 |

| | |
|---|---|
| Total Score | 0/120 |

### Excision Diabetic Wound Model

The in vivo model healing potential of freeze-dried Berberis extract loaded SLNs gel and Berberis extract gel was assessed in comparison to marketed product (Soframycin: 1% w/w) using excision wound model. Representative images of the reduction in the excision wound size with the passage of time (at 0, 3, 7, 14, 18 days) are depicted in FIG. 19. The disease control, control (wound only), blank SLNs gel showed delay in wound healing as compared to other groups. In disease control group, typical infectious diabetic wounds and delayed pattern of wound healing was observed, whereas, in control (wound only) some healing was observed. The wound healing in animals receiving freeze-dried Berberis extract loaded SLNs gel application was better (97% wound healing) than the animal receiving Berberis extract gel application (90% wound healing). Biochemical estimations and histopathology studies were also performed to study the healing process.

TABLE 5
Wound healing activity (diameter of wound in mm)

| | Freeze-dried | | | | |
|---|---|---|---|---|---|
| | Berberis | | | | |
| | extract loaded | Berberis | Blank | Marketed | Disease |
| Day | SLN gel | extract gel | SLNs gel | standard | control |
| 0 | 5.2 ± 0.08 | 5.0 ± 0.11 | 5.0 ± 0.08 | 5.2 ± 0.09 | 5.1 ± 0.1 |
| | (0) | (0) | (0) | (0) | |
| 3 | 4.65 ± 0.09 | 4.8 ± 0.08 | 4.9 ± 0.05 | 4.75 ± 0.07 | 5.8 ± 0.11 |
| | (10.57) | (4) | (2) | (8.65) | |
| 7 | 2.8 ± 0.04 | 3.17 ± 0.06 | 4 ± 0.07 | 3.13 ± 0.06 | 8 ± 0.12 |
| | (46.15) | (36.6) | (20) | (37.5) | |
| 14 | 0.75 ± 0.05 | 0.85 ± 0.04 | 1.37 ± 0.05 | 0.85 ± 0.03 | 10 ± 0.18 |
| | (85.57) | (83) | (72.5) | (83.65) | |
| 18 | 0.15 ± 0.02 | 0.5 ± 0.03 | 1 ± 0.03 | 0.45 ± 0.02 | 7.5 ± 0.15 |
| | (97.11) | (90) | (80) | (91.34) | |

*The values in parenthesis indicates percent wound healing

### Incision Diabetic Wound Model

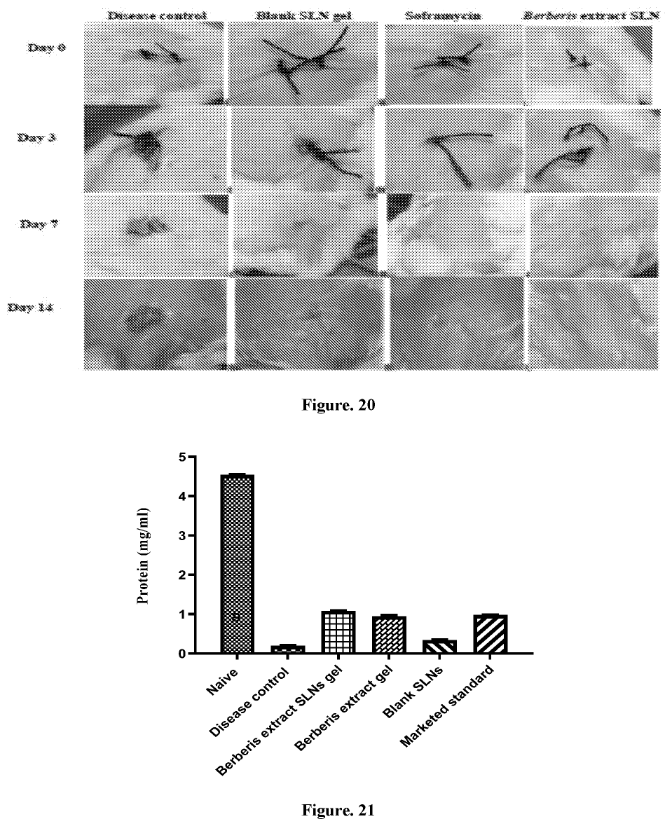

The in vivo healing potential of the freeze-dried Berberis extract loaded SLNs gel was assessed in comparison with the commercial products (Soframycin: 1% w/w) using incision diabetic wound model for 14 days. The representative images of incision wound healing with passage of time (0, 3, 7, 14 days) are depicted in FIG. 20. The disease control and blank SLNs gel group demonstrated delayed healing process compared to other groups. Faster re-modelling was observed in case of groups treated with freeze-dried Berberis extract loaded SLNs gel and marketed standard group. Biochemical estimations and histopathology studies were also performed to study the healing process.

### Example 16

### Biochemical Investigations

### Protein Estimation

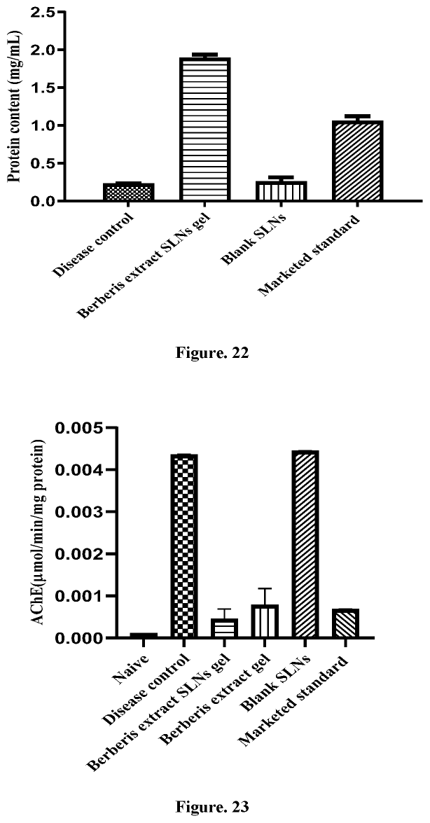

Protein plays central role in wound healing through the production of collagen. Hence an increase in protein content confirms the healing of wounds. Protein content was significantly decreased in disease control as compared to naïve group. All treatment groups in diabetic wound excision model showed significantly different activity from disease control group as depicted in FIG. 21. Protein content increased by 31.9% in case of freeze-dried Berberis extract loaded SLNs gel, by 27% in case of Berberis extract gel and 28% for marketed standard (p<0.05) compared to disease control group. Blank SLNs gel showed protein content increase by 5.2% as compared to disease control group. In a diabetic wound, freeze-dried Berberis extract loaded SLNs gel showed significantly different protein content versus Berberis extract gel by 5%. In diabetic wound incision model, treatment groups showed significantly different activity from disease control group as depicted in FIG. 22. Protein content increased by 34.14% in case of freeze-dried Berberis extract loaded SLNs gel and 17.07% for marketed standard (p<0.05) compared to disease control group. Blank SLNs gel showed protein content increase by 0.62% compared to disease control group.

### Acetylcholinesterase Inhibitory Activity

Acetylcholine plays a role in wound epithelialization. Acetylcholinesterase (AChE) is responsible for breakdown of acetylcholine so, decrease in levels of acetylcholinesterase confirms healing of wounds.

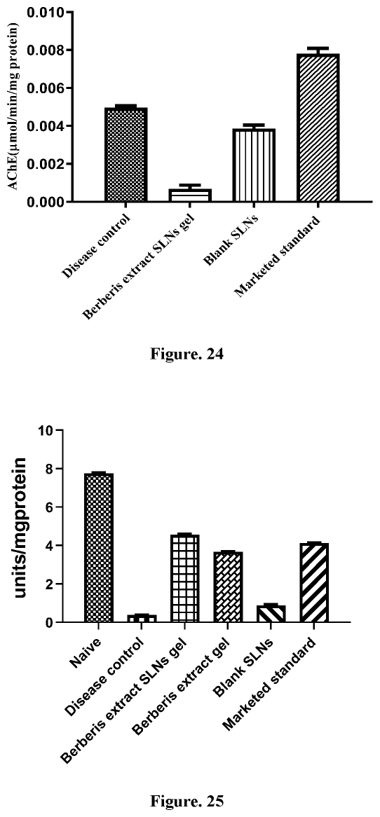

AChE significantly increased in disease control as compared to naïve group. All treatment groups in diabetic wound excision model showed significantly different activity from disease control group as depicted in FIG. 23. AChE decreased by 21.62% in case of freeze-dried Berberis extract loaded SLNs gel, 19.05% in Berberis extract gel and 21.22% for marketed standard (p<0.05) compared to disease control group. Blank SLNs gel showed AChE decreased by 0.5% as compared to disease control group. Freeze-dried Berberis extract loaded SLNs gel showed significantly different AChE levels versus Berberis extract gel. All treatment groups in diabetic wound incision model showed significantly different activity from disease control group as depicted in FIG. 24. AChE decreased by 25.64% in case of freeze-dried Berberis extract loaded SLNs gel and 26.95% for marketed standard (p<0.05) compared to disease control group. Blank SLNs gel showed AChE decrease by 6% as compared to disease control group.

### Measurement of Free Radical and Antioxidants Level

Free radicals (lipid peroxidation), antioxidants (catalase, superoxide dismutase and reduced glutathione) imbalance in free radical generation and antioxidants induce oxidative stress, tissue damage and delayed wound healing. So, decrease in free radicals and increase in antioxidants content helps in chronic wound healing. The superoxide dismutase (SOD), glutathione (GSH) and catalase levels were significantly decreased in disease control group in comparison to naïve group. All treatment groups in diabetic wound excision model showed significantly different activity from disease control group as depicted in FIGS. 25-27. SOD increased by 117.4% in case of freeze-dried Berberis extract loaded SLNs gel and 105.3% for marketed standard (p<0.05) compared to disease control group. Freeze-dried Berberis extract loaded SLNs gel showed significantly different SOD levels versus Berberis extract gel by 24.94%. Blank SLNs gel showed an increase of 14.10% compared to disease control group. GSH increased by 21.77% in case of freeze-dried Berberis extract loaded SLNs gel and 17.02% in case of marketed standard (p<0.05) compared to disease control group. In case of Berberis extract gel, GSH increased by 8.25%, and blank SLNs gel showed an increase of 3.5% compared to disease control group. Freeze-dried Berberis extract loaded SLNs gel versus Berberis extract gel showed significantly different activity. Freeze-dried Berberis extract loaded SLNs gel showed increase by 13.52% in comparison to Berberis extract gel. Catalase increased by 68% in case of freeze-dried Berberis extract loaded SLNs gel and 49.7% in case of marketed standard (p<0.05) compared to disease control group. In case of Berberis extract gel, catalase increased by 38.25% and blank SLNs gel showed an increase of 6.85% compared to disease control group. Freeze-dried Berberis extract loaded SLNs gel versus Berberis extract gel showed significantly different catalase levels. Freeze-dried Berberis extract loaded SLNs gel exhibited a 29.77% increase in catalase as compared to Berberis extract gel.

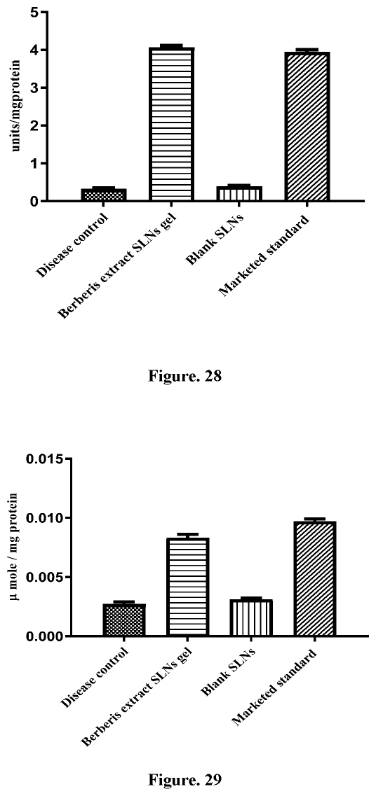

In case of diabetic wound incision model, all treatment groups showed significantly different activity from disease control group as depicted in FIGS. 28-30. SOD increased by 67.02% in case of freeze-dried Berberis extract loaded SLNs gel and 65.01% in case of marketed standard (p<0.05) compared to disease control group. In case of blank SLNs gel, increase by 1.12% as compared to disease control group was observed. GSH increased by 28.73% in case of freeze-dried Berberis extract loaded SLNs gel and 35.91% in case of marketed standard (p<0.05) compared to disease control group. In case of blank SLNs gel, increase of 2.05% compared to disease control group was observed. Catalase increased by 67.85% in case of freeze-dried Berberis extract loaded SLNs gel and 105.9% in case of marketed standard (p<0.05) compared to disease control group. In case of blank SLNs gel, a 6.85% increase compared to disease control group was observed.

The lipid peroxidation (LPO) levels significantly increased in disease control group in comparison to naïve group. All treatment groups in diabetic wound excision model showed significantly different activity from disease control group as depicted in FIG. 31. LPO level decreased by 51% in case of freeze-dried Berberis extract loaded SLNs gel and 49.15% in case of marketed standard (p<0.05) compared to disease control group. In case of Berberis extract gel, LPO decreased by 46.68% and blank SLNs gel showed a 19.22% decrease compared to disease control group.

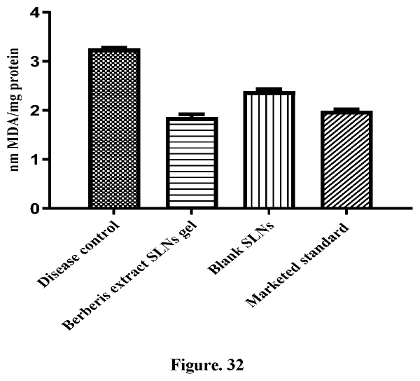

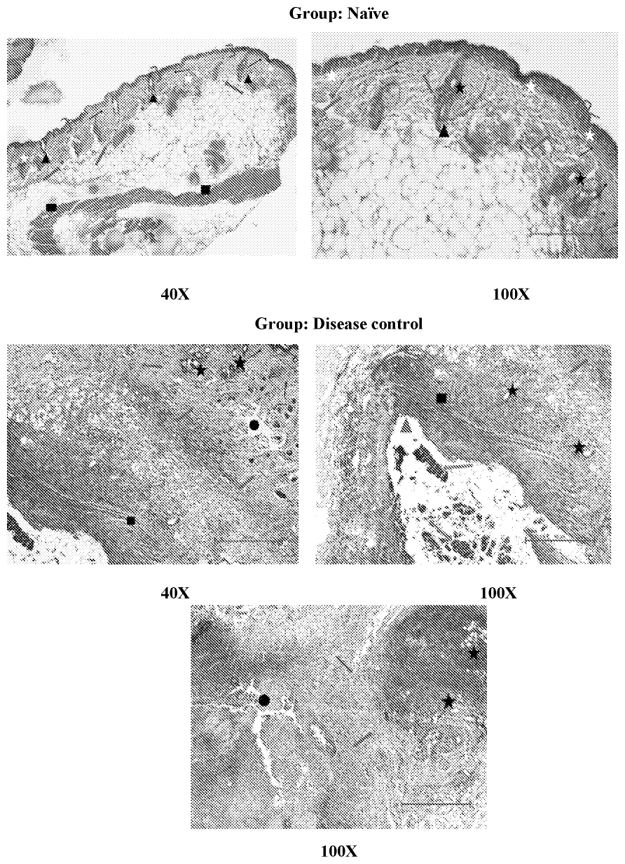

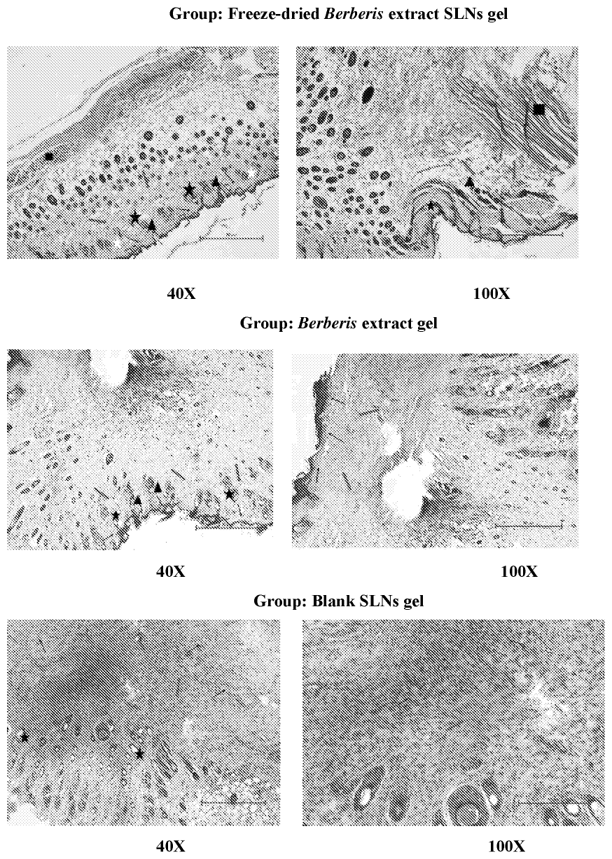

In case of diabetic wound incision model, LPO levels decreased by 32.32% in case of freeze-dried Berberis extract loaded SLNs gel and 29.3% in case of marketed standard (p<0.05) compared to disease control group as shown in FIG. 32. In case of blank SLNs gel, LPO levels decreased by 19.22% compared to disease control group.

### Example 17

### Histopathological Studies

### Effect of Wound Healing on Skin Tissue in Diabetic Wound Excision Model

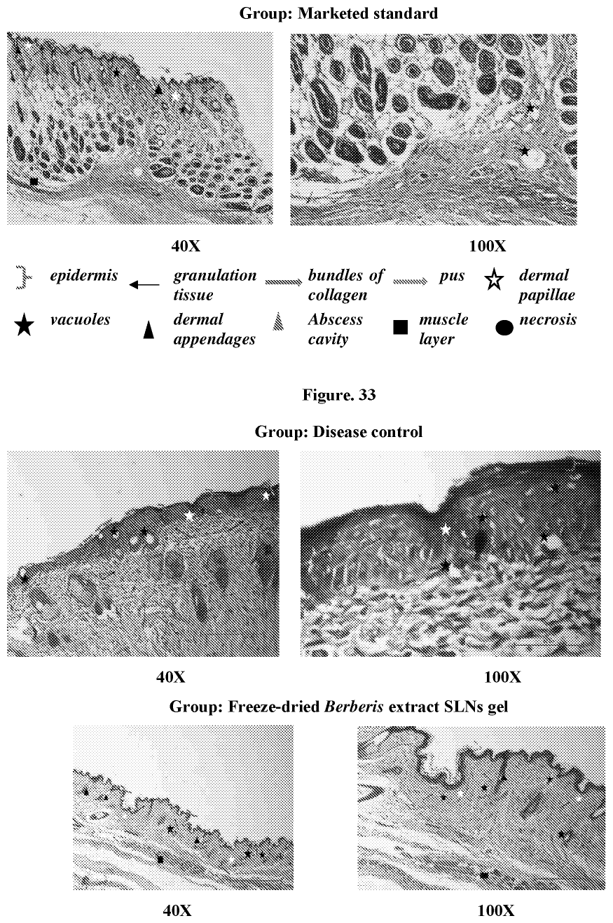

Hematoxylin and eosin-stained sections of skin are shown in FIG. 33. Histopathological study of naïve group showed normal skin from epidermis to subcutaneous tissue. Epidermis was well organized in multiple cell layers, along with numerous dermal papillae. Presence of bundles of collagen fibres and dermal appendages was also observed. However, in case of disease control eosinophil necrotic tissue, abscess cavity containing pus was observed. In case of blank SLNs gel, active inflammation with signs of early healing was observed.

In case of treatment groups, freeze-dried Berberis extract loaded SLNs gel and marketed standard, a partial or full healing of skin was observed as compared to other groups. In case of freeze-dried Berberis extract loaded SLNs gel, healing in upper part of dermis was observed. Proliferation of fibroblasts, collagen fibres began to reform and presence of vacant dermal appendages was observed. In case of marketed standard group, epidermis and dermis were fairly normal and mild edema in the deeper dermis was observed.

While in case of Berberis extract gel, fibroblasts maturing into collagen were observed. There were signs of re-epithelialization and presence of scattered collagen fibres were observed. So, in conclusion, excellent healing was observed in freeze-dried Berberis extract loaded SLNs gel and at the end of treatment, normal skin was observed in comparison to other treatment groups. Disease control showed the condition of typical diabetic wounds with infectious abscess cavity containing pus and delayed wound healing pattern.

### Effect of Wound Healing on Skin Tissue in Diabetic Wound Incision Model

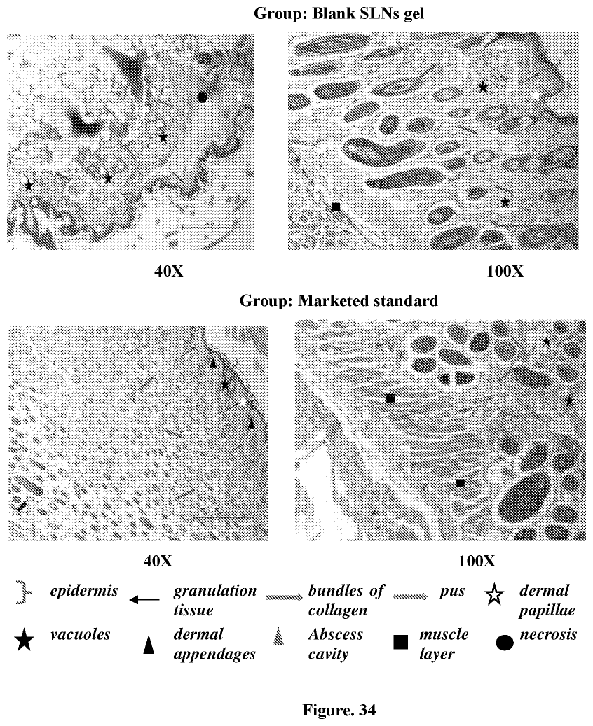

Hematoxylin and eosin-stained sections of skin for incision model are shown in FIG. 34. Histopathological study of disease control group showed irregular epidermis with focal hyperplasia, with fair amount of chronic inflammatory cells and fibroblasts in the dermis. In deep subcutaneous tissue, edema was seen with fibroblast proliferation and inflammation. In case of blank SLNs gel, linear healing scar from the epidermis was seen along with presence of degenerating necrotic tissue and localized foci of hyperplasia was also observed.

In case of treatment groups freeze-dried Berberis extract loaded SLNs gel and marketed standard drug, there were signs of healing of tissue. In freeze-dried Berberis extract loaded SLNs gel, intact irregular epidermis was observed. There was presence of fibrous healing and muscle fibers. In case of marketed standard, hair follicles were normal and early fibrosis in the subcutaneous tissue beneath muscle layer was observed.

### In-Vitro Anti-Inflammatory Activity of Berberis Extract

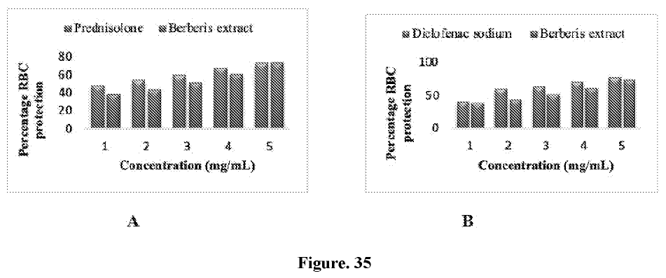

The percentage of RBC membrane protection by Berberis extract was compared against diclofenac sodium (non-steroidal) and prednisolone (steroidal) anti-inflammatory drugs. The activity was observed to be concentration dependent. Data showed that higher the concentration of Berberis extract, greater is the RBC membrane protection. The maximum protection of 73% was seen at concentration of 5 mg/mL. The results were compared with standards diclofenac sodium and prednisolone which also showed increase in RBC with increase in concentration to 5 mg/mL. The respective protection of 76% and 73% at concentration of 5 mg/mL was observed with these two standard drugs. This showed that the Berberis extract has significant anti-inflammatory activity which was comparable to standard drugs as depicted in FIG. 35.

1) A lipid nano-formulation comprising freeze-dried Berberis extract and at least one lipid.

2) The lipid nano-formulation as claimed in claim 1, wherein the formulation comprises:
a) freeze-dried Berberis extract in a range of 0.5-10 percent by weight,
b) at least one lipid in a range of 2-12 percent by weight,
c) at least one surfactant in a range of 2-10 percent by weight,
d) at least one cosurfactant in a range of 0.2-1 percent by weight,
e) at least one solubilizing agent in a range of 4-10 percent by weight,
f) water,
wherein the percent by weight is with reference to the weight of the total formulation.

3) The lipid nano-formulation as claimed in claim 1, wherein the freeze-dried Berberis extract is an alcoholic, hydroalcoholic or aqueous extract of freeze-dried Berberis.

4) The lipid nano-formulation as claimed in claim 1, wherein the lipid component is selected from the group consisting of Compritol 888 ATO, stearic acid, glycerol monostearate, and Precirol.

5) The lipid nano-formulation as claimed in claim 2, wherein the solubilizing agent is selected from polyethylene glycol (PEG), polyvinylpyrrolidone (PVP), polyvinyl alcohol (PVA), glycerol, transcutol, labrafac, gelucire, hydrogenated vegetable glycerides, glyceryl citrate, glyceryl lactate, glyceryl lincolate, glyceryl oleate, polyglyceryl-4-cocoate, polyglyceryl-3-caprate and caprylate and their derivatives, polypropylene glycol, and propylene glycol.

6) The lipid nano-formulation as claimed in claim 2, wherein the surfactant is selected from the group consisting of ethylene oxide copolymers, propylene oxide copolymers, poloxamers, sorbitan ethylene oxide/propylene oxide copolymers, polysorbate 20, polysorbate 60, polysorbate 80, sorbitan esters, span 20, span 40, span 60, span 80, alkyl aryl polyether alcohol polymers, tyloxapol, bile salts, cholate, glycocholate, taurocholate, taurodeoxycholate, gemini surfactants, alcohols, diethylene glycol monoethyl ether, propanediol, capryl glucoside, decyl glucoside, kolliwax or mixtures thereof.

7) The lipid nano-formulation as claimed in claim 2, wherein the cosurfactant is selected from the group consisting of phospholipon 90 G, soy lecithin, egg lecithin, phosphatidylcholine, cholate, glycocholate, taurocholate, taurodeoxycholate, or mixtures thereof.

8) The lipid nano-formulation as claimed in claim 1, wherein the formulation comprises:
a) freeze-dried Berberis extract in a range of 0.5-10 percent by weight,
b) Compritol in a range of 2-12 percent by weight,
c) Polysorbate 80 in a range of 2-10 percent by weight,
d) Phospholipon 90 G in a range of 0.2-1 percent by weight,
e) PEG 400 in a range of 4-10 percent by weight,
f) water,
wherein the percent by weight is with reference to the weight of the total formulation.

9) The nano-formulation as claimed in claim 1, wherein the nano-formulation has a freeze-dried Berberis extract loading of 20 to 30% with respect to the lipid phase, freeze-dried Berberis extract entrapment efficiency in a range of 88-92%, particle size in a range of 50-500 nm and PDI in a range of 0.27-0.29.

10) The lipid nano-formulation as claimed in claim 1, wherein the lipid nano-formulation is solid lipid nanoparticles.

11) The lipid nano-formulation as claimed in claim 1, wherein the lipid nano-formulation is combined with a suitable excipient to result in a gel, a hydrogel, an organogel, a syrup, a paste, a cream, a liquid wash, a facewash, a mouthwash, an oral rinse, an ointment, a liquid ampoule, a dispersion, a nasal drops or spray, an aerosol spray, a powder, an orthotic aid, a liquid oral formulation, a facemask, a film, an implant, a tablet, lozenges, capsules, suppositories, pessaries, patches and gummies for oral, parenteral, dental, buccal, ocular, intranasal, vaginal, rectal, otic, transdermal and topical delivery.

12) The lipid nano-formulation as claimed in claim 1, wherein the formulation is as and when used for treating diabetes and diabetes associated complications, as a wound healer, as an antimicrobial, as an anti-infective, as an anti-inflammatory agent and as an antioxidant.

13) A process for preparation of the lipid nano-formulation of freeze-dried Berberis extract as claimed in claim 1 comprising:
a) preparing a lipid phase by melting one or more lipid at a temperature at least equal to the melting point of said one or more lipid,
b) separately preparing an aqueous phase by heating at least one emulsifier or cosurfactant, at least one surfactant, and water at a temperature at least equal to said melting point of said one or more lipid of step a),
c) dissolving freeze-dried Berberis extract with at least one solubilizing agent to obtain a solution of freeze-dried extract of Berberis,
d) adding the solution of freeze-dried extract of Berberis to the lipid phase to obtain a hot lipid phase,
e) blending the hot lipid phase and the aqueous phase at a high speed of 5000-20,000 rpm for 5-30 minutes to obtain a hot emulsion,
f) subjecting hot emulsion obtained in step e) to two to six cycles of homogenization at 500-1200 bars pressure to obtain the lipid nano-formulation which is finally refrigerated after cooling to room temperature.

14) A process for preparing the freeze-dried Berberis extract as claimed in claim 1, comprising:
i. cutting and crushing plant parts to obtain a moderately fine powder of Berberis, wherein the plant part is a stem, root, or a mixture thereof and is from one of the species of genus Berberis or Mahonia,
ii. packing the powder obtained in step i. in a polyethylene bag and storing in a freezer at −20° C. to obtain frozen Berberis powder,
iii. carrying out reflux extraction of the frozen Berberis powder with an extraction solvent at 50-90° C. for 30 minutes to 8 hours,
iv. filtering the extracts through Whatman filter paper and concentrating the extract to dryness,
v. standardizing the extract by analysing the marker content of berberine, palmatine, jatrorrhizine and magnoflorine, producing fingerprint profiles and ATR fingerprints to obtain a standardized extract of Berberis,
vi. pre-treating the standardized extract by concentrating the extract at a temperature in a range of 19° C. to 70° C. and pressure in a range of 10 mmHg/torr to 30 mmHg/torr for reducing extract volume to about 1 to 25 mL to obtain concentrated extract,
vii. freezing the extract obtained in step vi. at a temperature in a range of −30 to −50° C. for 8 to 24 hours with 1-10 mm thickness of concentrated extract,
viii. primary drying the extract obtained in step vii. at a temperature in a range of −45 to −50° C. and pressure in a range of 25 to 30 mTorr for 15 to 20 hours, and
ix. secondary drying the extract obtained in step viii. at a temperature in a range of −45 to −60° C. and pressure in a range of 25 to 30 mTorr for 15 to 48 hours to obtain the freeze-dried Berberis extract.

15) The process as claimed in claim 14, wherein the extraction solvent of step iii. is an organic or inorganic solvent.

16) The process as claimed in claim 15, wherein the extraction solvent of step iii. is an alcohol, water or a mixture of alcohol and water.
